(19) United States
(12) Patent Application Publication
(10) Pub. No.: US 2016/0346408 A1
(43) Pub. Date: Dec. 1, 2016

(54) IRON STABILIZED MICELLES AS MAGNETIC CONTRAST AGENTS

(71) Applicant: Intezyne Technologies, Tampa, FL (US)

(72) Inventors: David Kelsen, Teaneck, NJ (US); Kevin Sill, Tampa, FL (US)

(21) Appl. No.: 15/165,707

(22) Filed: May 26, 2016

Related U.S. Application Data
(60) Provisional application No. 62/266,161, filed on Dec. 11, 2015.
(60) Provisional application No. 62/166,498, filed on May 26, 2015.

(51) Int. Cl. A61K 49/18; A61K 9/107

(52) U.S. Cl. CPC A61K 49/1809; A61K 9/1075

(57) ABSTRACT

The present invention relates to the field of polymer chemistry and more particularly to multiblock copolymers and iron stabilized micelles comprising the same, as magnetic contrast agents. Compositions herein are useful for diagnostic and drug-delivery applications.

### CROSS-REFERENCE TO RELATED APPLICATIONS

This application claims the benefit of U.S. Provisional Application No. 62/266,161, filed Dec. 11, 2015, and U.S. Provisional Application No. 62/166,498, filed May 26, 2015, the entirety of which is incorporationed by reference.

### FIELD OF THE INVENTION

The present invention relates to the field of polymer chemistry and more particularly to multiblock copolymers and uses thereof.

### BACKGROUND OF THE INVENTION

Although bones are easily visualized using x-ray imaging, many other organs and tissues cannot be easily imaged without contrast enhancement. Contrast agents, also known as contrast media or diagnostic agents, are often used during medical imaging examinations to highlight specific parts of the body (e.g. tissues and organs) and make them easier to visualize and improve disease diagnosis. Contrast agents can be used with many types of imaging examinations, including x-ray exams, computed tomography scans, magnetic resonance imaging, and positron emission tomography to name but a few.

### BRIEF DESCRIPTION OF THE DRAWINGS

FIG. 1. Schematic illustration depicting a drug loaded, iron stabilized micelle FIG. 2. Phantom imaging results of non-drug loaded, iron stabilized micelles. FIG. 2a depicts T1 relaxation results and FIG. 2b depicts T2 relaxation results.

FIG. 3. Phantom imaging results of SN-38 loaded, iron stabilized micelles. FIG. 3a depicts T1 relaxation results and FIG. 3b depicts T2 relaxation results.

FIG. 4. T1 weighted MRI images (transverse view; cross sections) of HCT-116 human colon carcinoma xenograft mouse prior to dosing.

FIG. 5. T1 weighted MRI images (transverse view; cross sections) of HCT-116 human colon carcinomaxenograft mouse 2.5 hours after dosing with SN-38 loaded, iron stabilized micelles.

FIG. 6. T1 weighted MRI images (transverse view; cross sections) of HCT-116 human colon carcinoma xenograft mouse 5 hours after dosing with SN-38 loaded, iron stabilized micelles.

FIG. 7. T1 weighted MRI images (transverse view; cross sections) of HCT-116 human colon carcinoma xenograft mouse 20 hours after dosing with SN-38 loaded, iron stabilized micelles.

FIG. 8. T1 weighted MRI images (transverse view, cross sections) of HCT-116 human colon carcinoma xenograft mouse 24 hours after dosing with SN-38 loaded, iron stabilized micelles.

FIG. 9. T1 weighted MRI images (transverse view, cross sections) of HCT-116 human colon carcinoma xenograft mouse 168 hours after dosing with SN-38 loaded, iron stabilized micelles.

FIG. 10. T2 weighted MRI images (transverse view, cross sections) of HCT-116 human colon carcinoma xenograft mouse prior to dosing.

FIG. 11. T2 weighted MRI images (transverse view, cross sections) of HCT-116 human colon carcinoma xenograft mouse 2.5 hours after dosing with SN-38 loaded, iron stabilized micelles.

FIG. 12. T2 weighted MRI images (transverse view, cross sections) of HCT-116 human colon carcinoma xenograft mouse 5 hours after dosing with SN-38 loaded, iron stabilized micelles.

FIG. 13. T2 weighted MRI images (transverse view, cross sections) of HCT-116 human colon carcinoma xenograft mouse 20 hours after dosing with SN-38 loaded, iron stabilized micelles.

FIG. 14. T2 weighted MRI images (transverse view, cross sections) of HCT-116 human colon carcinoma xenograft mouse 24 hours after dosing with SN-38 loaded, iron stabilized micelles.

FIG. 15. T2 weighted MRI images (transverse view, cross sections) of HCT-116 human colon carcinoma xenograft mouse 168 hours after dosing with SN-38 loaded, iron stabilized micelles.

FIG. 16. TEM image of HCT-116 human colon carcinoma xenograft tumor cross-section collected 1 hour after dosing with SN-38 loaded, iron stabilized micelles.

FIG. 17. TEM image of HCT-116 human colon carcinoma xenograft tumor cross-section collected 1 hour after dosing with SN-38 loaded, iron stabilized micelles.

FIG. 18. TEM image of HCT-116 human colon carcinoma xenograft tumor cross-section collected 1 hour after dosing with SN-38 loaded, iron stabilized micelles.

FIG. 19. T2 weighted MRI images (coronal view, cross sections) of HCT-116 human colon carcinoma xenograft mouse at different time points.

FIG. 20. A histogram comparing MRI contrast in tumor regions of interest (ROI) predose and at 24 hours.

FIG. 21. MR image (FIG. 21a) pre-dose and 48 hours post dosing of epothilone D loaded, iron stabilized micelles in lung cancer NCI-H460 xenograft mouse; MR image (FIG. 21b) pre-dose and 48 hours post dosing of epothilone D loaded, iron stabilized micelles in colon cancer HCT116 xenograft mouse. The tumor is in shown in the lower left of each image.

### DETAILED DESCRIPTION OF CERTAIN EMBODIMENTS OF THE INVENTION

### 1. General Description

Magnetic resonance imaging is useful in the medical field for imaging various tissues within a subject. The imaging process involves the use of a magnetic field to orient the spins of the nuclei of protons within water molecules. This orientation of spins “excites” the proton into a higher energy level. The proton then “relaxes” to the ground state, or equilibrium state, by emitting energy in the form of radio waves. The characteristic time of this relaxation contains information about the environment of the water molecules. Different tissues possess different relaxation times. For example, fatty tissue has a much shorter relaxation time than other tissues. The characteristic relaxation times can be combined to form an image.

Contrast agents are commonly utilized in medical imaging. In magnetic resonance imaging, such contrast agents typically shorten the relaxation time of protons in water molecules, causing them to relax much faster in the presence of the contrast agents. Due to the larger different in relaxation times, greater contrast can be observed in the resulting images through the use of contrast agents.

Magnetic nanoparticles, such as: Fe, Fe₂O₃, Fe₃O₄, MnFe₂O₄, CoFe₂O₄, NiFe₂O₄, Co, Ni, FePt, CoPt, CoO, Fe₃Pt, Fe₂Pt, Co₃Pt, Co₂Pt, FeOOH, have been useful for in vitro and in vivo diagnostics and treatments. Nanoparticles of this type, with sizes ranging from 2 nm-100 nm, have been successfully utilized as contrast agents for magnetic resonance, magnetically-controlled drug delivery vehicles, and in hyperthermia treatments. See: Jeong, U.; Teng, X.; Wang, Y.; Yang, H.; Xia, Y. “Superparamagnetic Colloids: Controlled Synthesis and Niche Applications” Adv. Mater.; 2007, 19, 33-60. Niederberger, M.; Garnweitner, G. “Organic Reaction Pathways in the Nonaqueos Synthesis of Metal Oxide Nanoparticles” 2006, 12, 7282-7302. Sun, S.; Zeng, H.; “Size-controlled Synthesis of Magnetite Nanoparticles” 2002, 124, 8204-8205.

Magnetic nanoparticles have been encapsulated in polymer micelles, including triblock copolymers, for use as contrast agents. See: U.S. patent application Ser. No. 12/112,799, published as 20090092554, on Apr. 9, 2009.

The hydrophobic forces that drive the aqueous assembly of colloidal drug carriers, such as polymer micelles and liposomes, are relatively weak, and these assembled structures dissociate below a finite concentration known as the critical micelle concentration (CMC). The CMC value of polymer micelles is of great importance in clinical applications because drug-loaded colloidal carriers are diluted in the bloodstream following administration and rapidly reach concentrations below the CMC (μM or less). This dilution effect will lead to micelle dissociation and drug release outside the targeted area and any benefits associated with the micelle size (enhanced permeability and retention, or EPR effect) or active targeting will be lost. While a great deal of research throughout the 1990's focused on identifying polymer micelles with ultra-low CMC values (nM or less), Maysinger (Savic et. al., Langmuir, 2006, p 3570-35′78) and Schiochet (Lu et. al., Macromolecules, 2011, p 6002-6008) have redefined the concept of a biologically relevant CMC by showing that the CMC values for polymer micelles shift by two orders of magnitude when the CMC values in saline are compared with and without serum.

Because drug-loaded micelles typically possess diameters greater than 20 nm, they exhibit dramatically increased circulation time when compared to stand-alone drugs due to minimized renal clearance. This unique feature of nanovectors and polymeric drugs leads to selective accumulation in diseased tissue, especially cancerous tissue due to the enhanced permeation and retention effect (“EPR”). The EPR effect is a consequence of the disorganized nature of the tumor vasculature, which results in increased permeability of polymer therapeutics and drug retention at the tumor site. In addition to passive cell targeting by the EPR effect, micelles are designed to actively target tumor cells through the chemical attachment of targeting groups to the micelle periphery. The incorporation of such groups is most often accomplished through end-group functionalization of the hydrophilic block using chemical conjugation techniques.

Despite the large volume of work on micellar drug carriers, only recently have efforts begun to focus on improving their in vivo stability to dilution. One potential reason is that the true effects of micelle dilution in vivo are not fully realized until larger animal studies are utilized. Because a mouse's metabolism is much higher than larger animals, they can receive considerably higher doses of toxic drugs when compared to larger animals such as rats or dogs. Therefore, when drug loaded micelles are administered and completely diluted throughout the entire blood volume, the corresponding polymer concentration will always be highest in the mouse model. Therefore, it would be highly desirable to prepare a micelle that is stabilized (crosslinked) to dilution within biological media. Furthermore, the EPR effect, the preference accumulation of nanoparticles in tumor tissue, requires an intact micelle (e.g. nanoparticles). Dissociation of the micelle results in premature release of the encapsulated therapeutic and leads to a biodistribution, efficacy, and toxicity profile similar to that of the free drug.

Previous work has utilized triblock copolymers containing carboxylic acids and/or hydroxamic acids to interact with metal ions in order to provide micelle stability. See: U.S. patent application Ser. No. 11/396,872, published as 20060240092 on Oct. 26, 2006; U.S. Ser. No. 13/839,715, published as 20130296531 on Nov. 7, 2013; and U.S. Ser. No. 13/621,652 published as 20130078310 on Mar. 28, 2013. Specifically, iron has been identified as a preferred metal ion for stabilization of triblock polymer micelles.

Iron ions and iron chelates generally do not exhibit superparamagnetic properties, precluding them from use as contrast agents in magnetic imaging. However, the iron oxide nanoparticles (Fe₂O₃, Fe₃O₄) described above possess superparamagnetic properties. The magnitude of the inherent paramagnetism in these nanoparticles is dependent upon particle size. It has been surprisingly found that the iron used to stabilize polymer micelles can act as a contrast agent in magnetic resonance imaging (MRI), allowing the direct imaging of drug loaded, iron stabilized micelles. Without wishing to be bound to any particular theory, it is believed that the spatial orientation of iron, a spherical shell in the outer core of the micelle imparts a paramagnetic or superparamagnetic effect, allowing the drug loaded, stabilized micelle to function as its own contrast agent. One skilled in the art will recognize that the iron ions have associated waters of coordination or solvation.

According to one embodiment, the present invention provides a drug loaded, iron stabilized micelle that provides contrast in magnetic imaging. Another embodiment of the present invention provides a method of monitoring the accumulation of drug loaded, iron stabilized micelles by magnetic resonance imaging (MRI). Another embodiment of the present invention provides a method of monitoring the accumulation of iron stabilized micelles by magnetic resonance imaging (MRI).

In certain embodiments, the present invention provides a method for imaging at least one tissue in a subject said method comprising administering to said subject a provided drug loaded, iron stabilized micelles, or composition thereof, and detecting said micelles by MRI.

In certain embodiments, the present invention provides a diagnostic imaging method comprising the steps of: (a) administering to a subject a provided iron stabilized micelles, or composition thereof; and (b) imaging the iron stabilized micelles after administration to the subject by magnetic resonance imaging.

In certain embodiments, the present invention provides a method of imaging at least one tissue in a subject comprising administering a provided iron stabilized micelles, or composition thereof, and performing an imaging procedure.

In certain embodiments, the subject is an animal. In certain embodiments, the animal is a mammal. In certain embodiments, the mammal is a primate. In certain embodiments, the primate is a human.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “contrast agent” (also known as “contrast media” and “radiocontrast agents”) refers to a compound used to improve the visibility of internal bodily structures during imaging.

As used herein, the term “T1” refers to spin-lattice relaxation time.

As used herein, the term “T2” refers to spin-spin relaxation time.

As used herein, the term “paramagnetism”, “paramagnetic”, “superparamagnetic” and “superparamagnetism” refers to a form of magnetism that is induced by an external magnetic field.

As used herein, the term “magnetic resonance imaging”, “nuclear magnetic resonance imaging”, “magnetic resonance tomography”, “MRT”, and “MRI” refer to a medical imaging technique that images tissues through the protons in water molecules.

As used herein, the terms “phantom image” or “phantom imaging” refer to the use of, or using, a non-living object containing a contrast medium, or media, at various concentrations, to evaluate, analyze, calibrate, and/or tune the performance of an imaging device.

As used herein, the term “voxel” refers to a representation of a value on a regular grid in three-dimensional space; a volume element, or three-dimensional analogue of a pixel.

As used herein, the term “SEMS” refers to a spin echo multislice pulse sequence.

As used herein, the term “MEMS” refers to a multiple echo multi shot pulse sequence.

As used herein, the term “ROI” means region of interest.

As used herein, the term “TEM” means transmission electron microscope or microscopy.

As used herein, the term “multiblock copolymer” refers to a polymer comprising one synthetic polymer portion and two or more poly(amino acid) portions. Such multi-block copolymers include those having the format W-X-X′, wherein W is a synthetic polymer portion and X and X′ are poly(amino acid) chains or “amino acid blocks”. In certain embodiments, the multiblock copolymers of the present invention are triblock copolymers. As described herein, one or more of the amino acid blocks may be “mixed blocks”, meaning that these blocks can contain a mixture of monomers thereby creating multiblock copolymers of the present invention. In some embodiments, the multiblock copolymers of the present invention comprise a mixed amino acid block and are tetrablock copolymers.

One skilled in the art will recognize that a monomer repeat unit is defined by parentheses around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by [(A)₄(B)₄(C)₄(D)₄].

As used herein, the monomer repeat unit described above is a numerical value representing the average number of monomer units comprising the polymer chain. For example, a polymer represented by (A)₁₀ corresponds to a polymer consisting of ten “A” monomer units linked together. One of ordinary skill in the art will recognize that the number 10 in this case will represent a distribution of numbers with an average of 10. The breadth of this distribution is represented by the polydispersity index (PDI). A PDI of 1.0 represents a polymer wherein each chain length is exactly the same (e.g. a protein). A PDI of 2.0 represents a polymer wherein the chain lengths have a Gaussian distribution. In some embodiments, a polymer of the present invention typically possessed a PDI of less than about 1.20.

As used herein, the term “triblock copolymer” refers to a polymer comprising one synthetic polymer portion and two poly(amino acid) portions.

As used herein, the term “inner core” as it applies to a micelle of the present invention refers to the center of the micelle formed by the hydrophobic D,L-mixed poly(amino acid) block. In accordance with the present invention, the inner core is not crosslinked. By way of illustration, in a triblock polymer of the format W-X′-X″, as described above, the inner core corresponds to the X″ block.

As used herein, the term “outer core” as it applies to a micelle of the present invention refers to the layer formed by the first poly(amino acid) block. The outer core lies between the inner core and the hydrophilic shell. In accordance with the present invention, the outer core interacts with iron to bind multiple polymers together. The linking of multiple polymers together with iron imparts stability to the micelle. By way of illustration, in a triblock polymer of the format W-X′-X″, as described above, the outer core corresponds to the X′ block. It is contemplated that the X′ block can be a mixed block.

As used herein, the terms “drug-loaded” and “encapsulated”, and derivatives thereof, are used interchangeably. In accordance with the present invention, a “drug-loaded” micelle refers to a micelle having a drug, or therapeutic agent, situated within the core of the micelle. In certain instances, the drug or therapeutic agent is situated at the interface between the core and the hydrophilic corona. This is also referred to as a drug, or therapeutic agent, being “encapsulated” within the micelle.

As used herein, the terms “crosslinked” and “stabilized” are used interchangeably. In accordance with the present invention, a “stabilized” micelle is comprised of a triblock copolymer and iron, wherein the iron interacts with the center block of the polymer to impart stability to the micelle.

As used herein, the term “polymeric hydrophilic block” refers to a polymer that is hydrophilic in nature. Such hydrophilic polymers are well known in the art and include polyethyleneoxide (also referred to as polyethylene glycol or PEG), and derivatives thereof, poly(N-vinyl-2-pyrolidone), and derivatives thereof, poly(N-isopropylacrylamide), and derivatives thereof, poly(hydroxyethyl acrylate), and derivatives thereof, poly(hydroxylethyl methacrylate), and derivatives thereof, and polymers of N-(2-hydroxypropoyl)methacrylamide (HMPA) and derivatives thereof.

As used herein, the term “polymeric stabilizing block” refers to a polymer that contains functionality that can interact (e.g. ligate or complex) with iron. Such functional groups include, but are not limited to, hydroxamic acid, carboxylic acid, catechols, amines, and nitrogen containing heterocycles.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit of the optionally crosslinkable or crosslinked poly(amino acid block) is in the L-configuration. Such poly(amino acids) include those having suitably protected functional groups. For example, amino acid monomers may have hydroxyl or amino moieties, which are optionally protected by a hydroxyl protecting group or an amine protecting group, as appropriate. Such suitable hydroxyl protecting groups and amine protecting groups are described in more detail herein, infra. As used herein, an amino acid block comprises one or more monomers or a set of two or more monomers. In certain embodiments, an amino acid block comprises one or more monomers such that the overall block is hydrophilic. In still other embodiments, amino acid blocks of the present invention include random amino acid blocks, i.e. blocks comprising a mixture of amino acid residues.

As used herein, the term “D,L-mixed poly(amino acid) block” refers to a poly(amino acid) block wherein the poly(amino acid) consists of a mixture of amino acids in both the D- and L-configurations. In certain embodiments, the D,L-mixed poly(amino acid) block is hydrophobic. In other embodiments, the D,L-mixed poly(amino acid) block consists of a mixture of D-configured hydrophobic amino acids and L-configured hydrophilic amino acid side-chain groups such that the overall poly(amino acid) block comprising is hydrophobic.

Exemplary poly(amino acids) include poly(benzyl glutamate), poly(benzyl aspartate), poly(L-leucine-co-tyrosine), poly(D-leucine-co-tyrosine), poly(L-phenylalanine-co-tyrosine), poly(D-phenylalanine-co-tyrosine), poly(L-leucine-coaspartic acid), poly(D-leucine-co-aspartic acid), poly(L-phenylalanine-co-aspartic acid), poly(D-phenylalanine-co-aspartic acid).

As used herein, the phrase “natural amino acid side-chain group” refers to the side-chain group of any of the 20 amino acids naturally occurring in proteins. For clarity, the side chain group —CH3 would represent the amino acid alanine. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyrosine nonpolar and hydrophobic by virtue of protecting the hydroxyl group.

As used herein, the phrase “unnatural amino acid side-chain group” refers to amino acids not included in the list of 20 amino acids naturally occurring in proteins, as described above. Such amino acids include the D-isomer of any of the 20 naturally occurring amino acids. Unnatural amino acids also include homoserine, ornithine, and thyroxine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, azidylated, labelled, and the like.

As used herein, the term “tacticity” refers to the stereochemistry of the poly(amino acid) hydrophobic block. A poly(amino acid) block consisting of a single stereoisomer (e.g. all L isomer) is referred to as “isotactic”. A poly(amino acid) consisting of a random incorporation of D and L amino acid monomers is referred to as an “atactic” polymer. A poly(amino acid) with alternating stereochemistry (e.g. . . . DLDLDL . . . ) is referred to as a “syndiotactic” polymer. Polymer tacticity is described in more detail in “Principles of Polymerization”, 3rd Ed., G. Odian, John Wiley & Sons, New York: 1991, the entire contents of which are hereby incorporated by reference.

The term hydroxamic acid, as used herein, refers to a moiety containing a hydroxamic acid (—CO—NH—OH) functional group. The structured is represented by

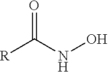

and may also be represented by

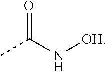

One skilled in the art would recognize that a dotted bond represents an attachment point to the rest of the molecule.

The term hydroxamate, as used herein, refers to a moiety containing either hydroxamic acid or an N-substituted hydroxamic acid. Due to the N-substitution, two separate locations exist for chemical attachment, as shown by the R and R′ groups here

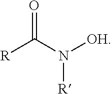

Hydoxamates may also be represented by

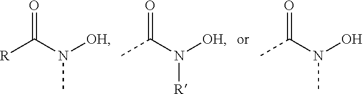

herein.

The term catechol, as used herein, refers to a substituted ortho-dihydroxybenezene derivative. Two different isomeric conformations are represented by

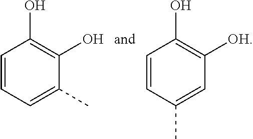

Catechol is also known as pyrocatechol and benzene-1,2-diol.

### 3. Description of Exemplary Embodiments

According to one embodiment, the present invention provides a micelle comprising a multiblock copolymer which comprises iron and a polymeric hydrophilic block, polymeric stabilizing block, and a polymeric hydrophobic block, characterized in that said micelle has an inner core, crosslinked outer core, and a hydrophilic shell. It will be appreciated that the polymeric hydrophilic block corresponds to the hydrophilic shell, the optionally crosslinkable or crosslinked polymeric block corresponds to the optionally crosslinked outer core, and the polymeric hydrophobic block corresponds to the inner core.

In certain embodiments, the present invention provides an iron stabilized micelle having an drug encapsulated therein, wherein said micelle comprises a multiblock copolymer which comprises:

a polymeric hydrophilic block;

a crosslinked outer core block; and

a polymeric hydrophobic block.

In other embodiments, the present invention provides an iron stabilized micelle wherein said micelle comprises a multiblock copolymer which comprises:

a polymeric hydrophilic block;

a crosslinked outer core block; and

a polymeric hydrophobic block,

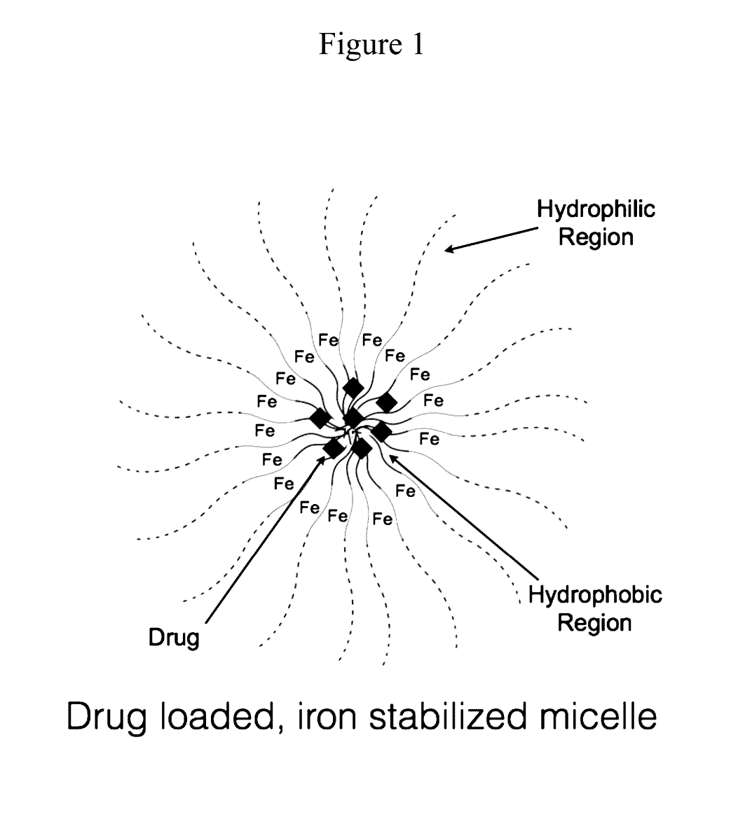

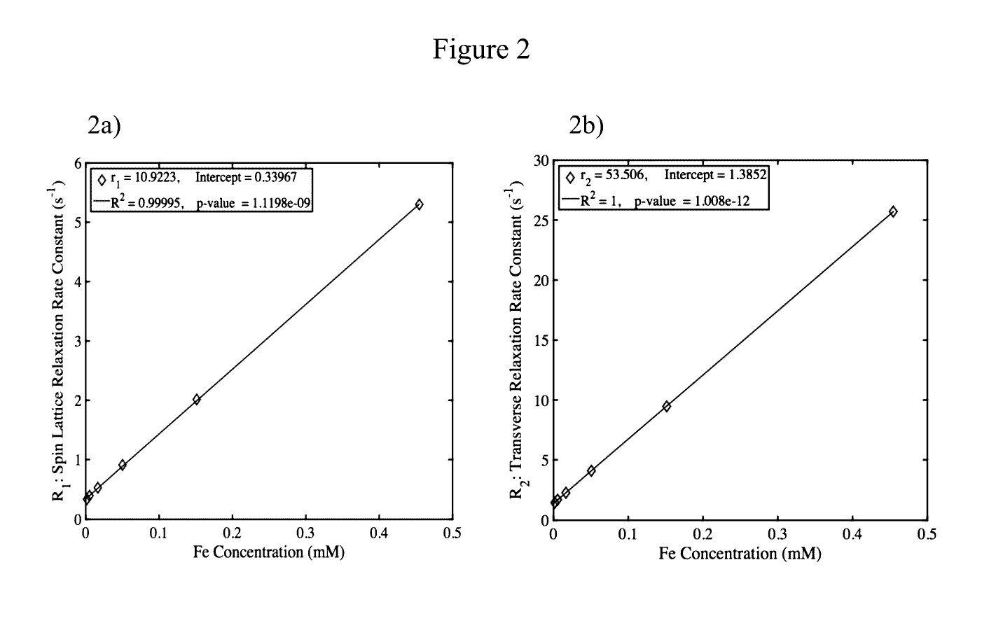

An illustration of drug loaded, iron stabilized micelles is provided in FIG. 1 of the drawings. It will be obvious to one skilled in the art that the drug loaded, stabilized micelle of the present invention is comprised of tens to thousands of polymer chains. It will be obvious to one skilled in the art that the drug loaded, stabilized micelle of the present invention is comprised of tens to millions of iron atoms. It will be obvious to one skilled in the art that the drug loaded, stabilized micelle of the present invention is comprised of tens to millions of drug molecules.

In oncology, it is highly desirable to predict the subject's response to a treatment prior to exposing the subject to the toxicity associated with many chemotherapies. Similarly, it is highly desirable to determine if the administered drugs are reaching the site of disease in a non-invasive manner. In some embodiments, the present invention provides a method of tracking the accumulation of drug loaded, iron crosslinked micelles (e.g. nanoparticles) using the inherent magnetic contrast of the iron used for stabilizing the micelle by MRI. The drug loaded, iron stabilized micelles are administered to the subject, then specific tissues within the subject imaged by MRI to determine if the nanoparticles are accumulating in the tissue of interest. A doctor may determine to amend the dose level or schedule based upon the results of these images. It is understood by one skilled in the art that the magnetic contrast imparted by the drug loaded, iron stabilized micelles is an inherent property of the micelle. For clarity, once the micelle is dissociated, without wishing to be bound to any particular theory, very little, if any magnetic contrast is present. One skilled in the art will further understand that any contrast observed in the MRI is a direct result of intact micelles.

In certain embodiments, a non-drug loaded, iron stabilized micelle may be used for diagnostic purposes. For clarity, no therapeutic benefit would be expected, but the non-drug loaded, iron stabilized micelle would possess utility as a contrast agent.

In certain embodiments, the present invention provides a diagnostic imaging method comprising the steps of: (a) administering to a subject a provided non-drug loaded, iron stabilized micelles, or composition thereof; and (b) imaging the iron stabilized micelles after administration to the subject by magnetic resonance imaging.

In oncology, cancer prevention and early detection is currently an unmet medical need. Without wishing to be bound to any particular theory, it is believed that non-drug loaded, iron stabilizied micells of the present invention possess utility in the detection of small sites of disease. Imaging of small sites of disease or small tumors will aid in the early detection of cancer.

In certain embodiments, the present invention provides a diagnostic imaging method comprising the steps of: (a) administering to a subject a provided non-drug loaded, iron stabilized micelles, or composition thereof; and (b) imaging the iron stabilized micelles after administration to the subject by magnetic resonance imaging, and (c) detecting the presence of a tumor or tumors within the subject.

Diagnostic imaging is an important aspect of staging of cancer patients. Staging (determinging the stage of the cancer) typically includes, but is not limited to, physical exams, imaging, diagnostic tests, and blood chemistry. The stage of the cancer is determined by a number of factors including: the size of the tumor, whether or not the tumor has metastasized, where the tumor is located, tumor cell type, and likelihood that the tumor will spread. Positron emmission tomography-computed tomography (PET-CT) is often used for imaging of tumors within subjects. However, there is radiation exposure associated with PET-CT scans. Therefore, it would be advantageous to utilize an imaging methodology without exposure to the radiation associated with PET-CT.

In certain embodiments, the present invention provides a diagnostic imaging method comprising the steps of: (a) administering to a subject a provided non-drug loaded, iron stabilized micelles, or composition thereof; and (b) imaging the iron stabilized micelles after administration to the subject by magnetic resonance imaging, and (c) determining the stage of cancer within the subject.

One skilled in the art will recognize that drug loaded, iron stabilized micelles of the present invention serve a dual purpose, both as a magnetic contrast agent (e.g. diagnostic) and as providing therapeutic benefit in the delivery of a drug. Such dual utility is sometimes referred to as a “theragnostic”.

In certain embodiments, the present invention provides a method for imaging at least one tissue in a subject, said method comprising administering to said subject a provided drug loaded, iron stabilized micelles, or composition thereof, and detecting said micelles by MRI.

In certain embodiments, the present invention provides a diagnostic imaging method comprising the steps of: (a) administering to a subject a provided drug loaded, iron stabilized micelles, or composition thereof; and (b) imaging the iron stabilized micelles after administration to the subject by magnetic resonance imaging.

In certain embodiments, the present invention provides a method of imaging at least one tissue in a subject comprising administering a provided drug loaded, iron stabilized micelles, or composition thereof, and performing an imaging procedure.

In certain embodiments, the present invention provides a method of treating a subject and imaging at least one tissue following the administration of iron stabilized micelles, or composition thereof, and performing an imaging procedure.

In certain embodiments, the present invention provides a method comprising the following steps: 1) administration of drug loaded, iron stabilized micelles, or composition thereof, to a subject; 2) imaging at least one tissue with MRI; 3) optionally adjusting treatment duration or dose level.

In certain embodiments, the present invention provides a method of treating a subject with cancer comprising the following steps: 1) administration of drug loaded, iron stabilized micelles, or composition thereof, to a subject possessing a solid tumor malignancy; 2) imaging said tumor with MRI; 3) confirming that contrast is observed in the tumor; and 4) continuing treatment schedule.

In certain embodiments, the present invention provides a diagnostic imaging method comprising the steps of: (a) administering to a subject a provided drug loaded, iron stabilized micelles, or composition thereof; and (b) imaging the iron stabilized micelles after administration to the subject by magnetic resonance imaging, and (c) determining the stage of cancer within the subject.

In certain embodiments, the present invention provides a diagnostic imaging method comprising the steps of: (a) administering to a subject a provided drug loaded, iron stabilized micelles, or composition thereof; and (b) imaging the iron stabilized micelles after administration to the subject by magnetic resonance imaging, and (c) detecting the presence of a tumor or tumors within the subject.

Amphiphilic multiblock copolymers, as described herein, can self-assemble in aqueous solution to form nano- and micron-sized structures. In water, these amphiphilic multiblock copolymers assemble by multi-molecular micellization when present in solution above the critical micelle concentration (CMC). Without wishing to be bound by any particular theory, it is believed that the hydrophobic poly(amino acid) portion or “block” of the copolymer collapses to form the micellar core, while the hydrophilic PEG block forms a peripheral corona and imparts water solubility. In certain embodiments, the multiblock copolymers in accordance with the present invention possess distinct hydrophobic and hydrophilic segments that form micelles. In addition, these multiblock polymers optionally comprise a poly(amino acid) block which contains functionality for crosslinking. It will be appreciated that this functionality is found on the corresponding amino acid side-chain.

According to one embodiment, the present invention provides a micelle comprising a triblock copolymer which comprises a polymeric hydrophilic block, optionally a crosslinkable or crosslinked poly(amino acid block), and a hydrophobic D,L-mixed poly(amino acid) block, characterized in that said micelle has an inner core, optionally a crosslinkable or crosslinked outer core, and a hydrophilic shell. As described herein, micelles of the present invention are especially useful for encapsulating therapeutic agents. In certain embodiments the therapeutic agent is hydrophobic.

Without wishing to be bound by any particular theory, it is believed that the accommodation of structurally diverse therapeutic agents within a micelle of the present invention is effected by adjusting the hydrophobic D,L-mixed poly(amino acid) block, i.e., the block comprising R^{y}. As discussed above, the hydrophobic mixture of D and L stereoisomers affords a poly(amino acid) block with a random coil conformation thereby enhancing the encapsulation of hydrophobic drugs.

Hydrophobic small molecule drugs suitable for loading into micelles of the present invention are well known in the art. In certain embodiments, the present invention provides a drug-loaded, iron stabilized micelle as described herein, wherein the drug is a hydrophobic drug selected from those described herein, infra.

As used herein, the terms hydrophobic small molecule drugs, small molecule drugs, therapeutic agent, and hydrophobic therapeutic agents are all interchangeable.

In certain embodiments, the present invention provides crosslinked micelles which effectively encapsulate hydrophobic or ionic therapeutic agents at pH 7.4 (blood) but dissociate and release the drug at targeted, acidic pH values ranging from 5.0 (endosomal pH) to 6.8 (extracellular tumor pH). In yet other embodiments, the pH value can be adjusted between 4.0 and 7.4. These pH-targeted nanovectors will dramatically improve the cancer-specific delivery of chemotherapeutic agents and minimize the harmful side effects commonly encountered with potent chemotherapy drugs. In addition, the utilization of chemistries which can be tailored to dissociate across a range of pH values make these drug-loaded micelles applicable in treating solid tumors and malignancies that have become drug resistant.

In other embodiments, the present invention provides a system comprising a triblock copolymer, a hydrophobic therapeutic agent, and iron. In another embodiment, comprising a triblock copolymer, a hydrophobic therapeutic agent, a cryoprotective agent and iron.

The ultimate goal of metal-mediated crosslinking is to ensure micelle stability when diluted in the blood (pH 7.4) followed by rapid dissolution and drug release in response to a finite pH change such as those found in a tumor environment.

In certain embodiments, the present invention provides a drug-loaded, iron stabilized micelle that provides contrast in magnetic resonance imaging, as described herein, wherein the drug is a taxane.

In certain embodiments, the present invention provides a drug-loaded, iron stabilized micelle that provides contrast in magnetic resonance imaging, as described herein, wherein the drug is paclitaxel.

In certain embodiments, the present invention provides a drug-loaded, iron stabilized micelle that provides contrast in magnetic resonance imaging, as described herein, wherein the drug is docetaxel.

In certain embodiments, the present invention provides a drug-loaded, iron stabilized micelle that provides contrast in magnetic resonance imaging, as described herein, wherein the drug is cabazitaxel.

In certain embodiments, the present invention provides a drug-loaded, iron stabilized micelle that provides contrast in magnetic resonance imaging, as described herein, wherein the drug is an epothilone.

In certain embodiments, the present invention provides a drug-loaded, iron stabilized micelle that provides contrast in magnetic resonance imaging, as described herein, wherein the drug is Epothilone D.

In certain embodiments, the present invention provides a drug-loaded, iron stabilized micelle that provides contrast in magnetic resonance imaging, as described herein, wherein the drug is Epothilone B.

In certain embodiments, the present invention provides a drug-loaded, iron stabilized micelle that provides contrast in magnetic resonance imaging, as described herein, wherein the drug is Epothilone A or Epothilone C.

In certain embodiments, the present invention provides a drug-loaded, iron stabilized micelle that provides contrast in magnetic resonance imaging, as described herein, wherein the drug is a vinca alkaloid.

In certain embodiments, the present invention provides a drug-loaded, iron stabilized micelle that provides contrast in magnetic resonance imaging, as described herein, wherein the drug is vinorelbine.

In certain embodiments, the present invention provides a drug-loaded, iron stabilized micelle that provides contrast in magnetic resonance imaging, as described herein, wherein the drug is berberine.

In certain embodiments, the present invention provides a drug-loaded, iron stabilized micelle that provides contrast in magnetic resonance imaging, as described herein, wherein the drug is berberrubine.

In certain embodiments, the present invention provides a drug-loaded, iron stabilized micelle that provides contrast in magnetic resonance imaging, as described herein, wherein the drug is a camptothecin.

In certain embodiments, the present invention provides a drug-loaded, iron stabilized micelle that provides contrast in magnetic resonance imaging, as described herein, wherein the drug is SN-38.

In certain embodiments, the present invention provides a drug-loaded, iron stabilized micelle that provides contrast in magnetic resonance imaging, as described herein, wherein the drug is S39625.

In certain embodiments, the present invention provides a drug-loaded, iron stabilized micelle that provides contrast in magnetic resonance imaging, as described herein, wherein the drug is an anthracycline.

In certain embodiments, the present invention provides a drug-loaded, iron stabilized micelle that provides contrast in magnetic resonance imaging, as described herein, wherein the drug is daunorubicin.

In certain embodiments, the present invention provides a drug-loaded, iron stabilized micelle that provides contrast in magnetic resonance imaging, as described herein, wherein the drug is doxorubicin.

In certain embodiments, the present invention provides a drug-loaded, iron stabilized micelle that provides contrast in magnetic resonance imaging, as described herein, wherein the drug is aminopterin.

In certain embodiments, the present invention provides a drug-loaded, iron stabilized micelle that provides contrast in magnetic resonance imaging, as described herein, wherein the drug is picoplatin.

In certain embodiments, the present invention provides a drug-loaded, iron stabilized micelle that provides contrast in magnetic resonance imaging, as described herein, wherein the drug is a platinum therapeutic.

Taxanes are well known in the literature and are natural products produced by plants of the genus Taxus. The mechanism of action is microtubule stabilization, thus inhibiting mitosis. Many taxanes are poorly soluble or nearly completely insoluble in water. Exemplary epothilones are shown below.

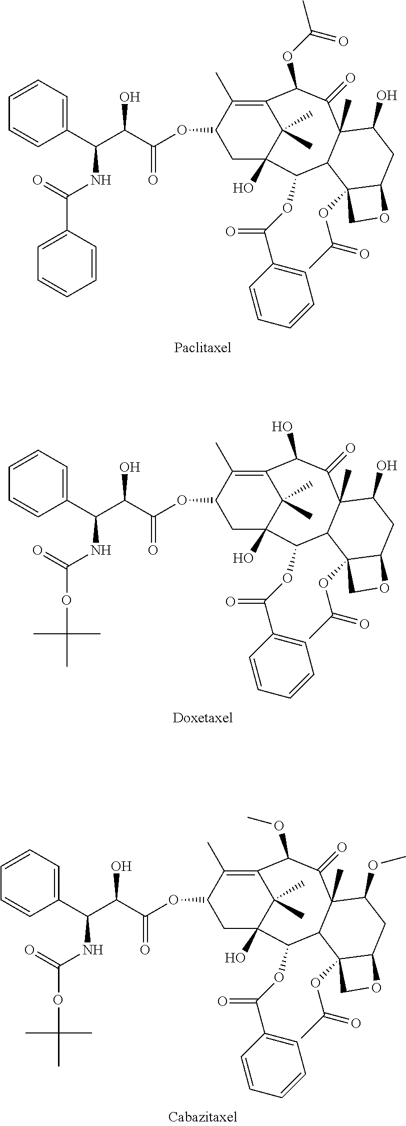

Epothilones are a group of molecules that have been shown to be microtubule stabilizers, a mechanism similar to paclitaxel (Bollag D M et al. Cancer Res. 1995, 55, 2325-2333). Biochemical studies demonstrated that epothilones can displace paclitaxel from tubulin, suggesting that they compete for the same binding site (Kowalski R J, Giannakakou P, Hamel E. J Biol Chem. 1997, 272, 2534-2541). One advantage of the epothilones is that they exert much greater cytotoxic effect in PGP overexpressing cells compared to paclitaxel. Exemplary epothilones are shown below.

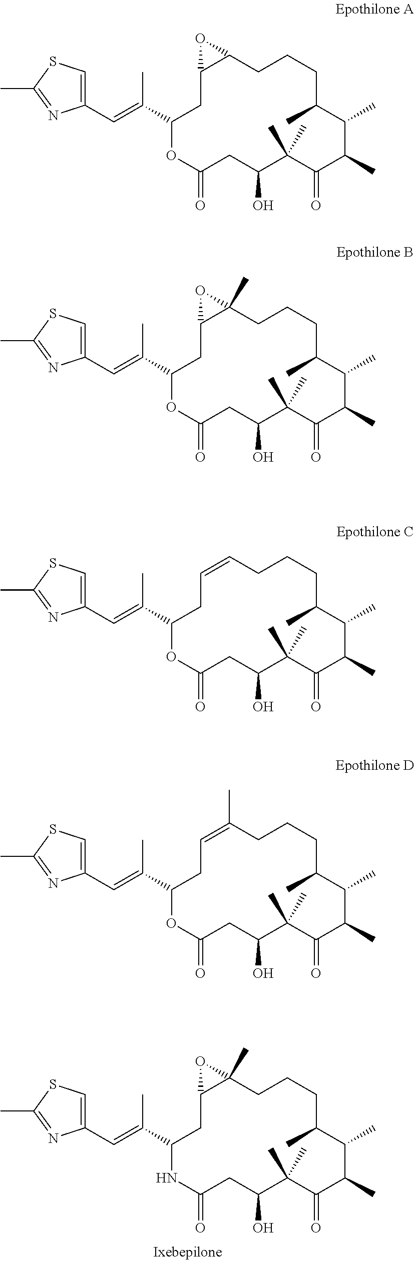

Vinca alkaloids are well known in the literature and are a set of anti-mitotic agents. Vinca alkaloids include vinblastine, vincristine, vindesine, and vinorelbine, and act to prevent the formation of microtubules. Exemplary vinca alkaloids are shown below.

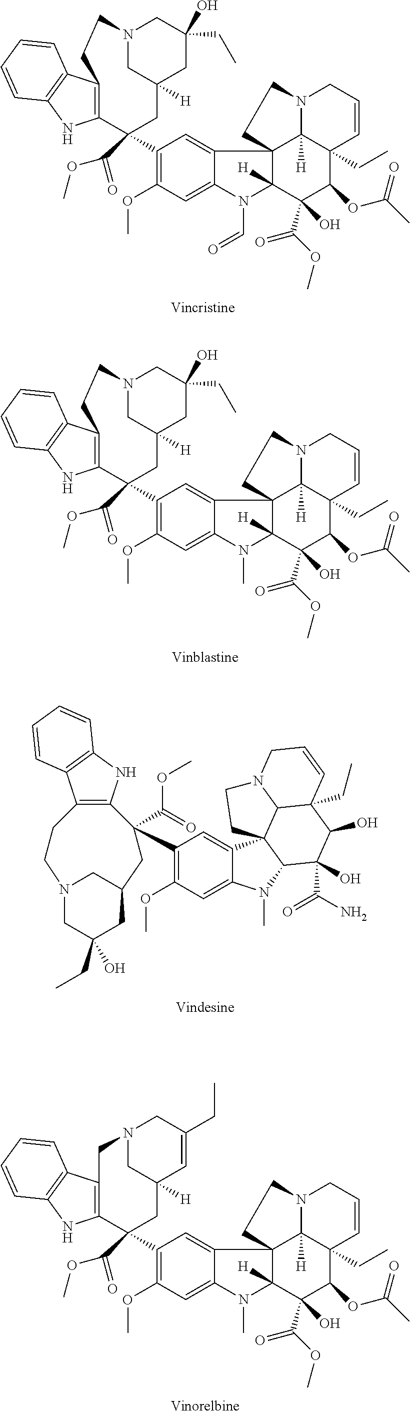

Berberine is well known in the literature and shown pharmaceutical effects in a range of applications including antibacterial and oncology applications. The anti-tumor activity of berberine and associated derivatives are described in Hoshi, et. al. Gann, 1976, 67, 321-325. Specifically, berberrubine and ester derivatives of berberrubine are shown to have increased anti-tumor activity with respect to berberine. The structures of berberine and berberrubine are shown below.

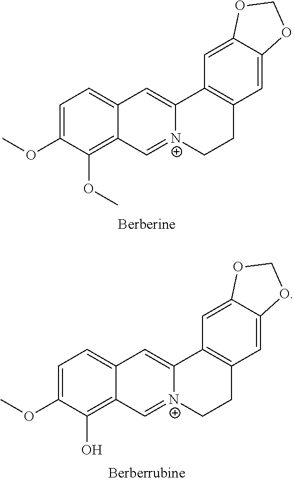 [SMILES: COc1ccc2cc3[n+](cc2c1OC)CCc1cc2c(cc1-3)OCO2 ; COc1ccc2cc3[n+](cc2c1O)CCc1cc2c(cc1-3)OCO2]

The antitumor plant alkaloid camptothecin (CPT) is a broad-spectrum anticancer agent that targets DNA topoisomerase I. Although CPT has shown promising antitumor activity in vitro and in vivo, it has not been clinically used because of its low therapeutic efficacy and severe toxicity. Among CPT analogues, irinotecan hydrochloride (CPT-11) has recently been shown to be active against colorectal, lung, and ovarian cancer. CPT-11 itself is a prodrug and is converted to 7-ethyl-10-hydroxy-CPT (known as SN-38), a biologically active metabolite of CPT-11, by carboxylesterases in vivo. A number of camptothecin derivatives are in development, the structures of which are shown below.

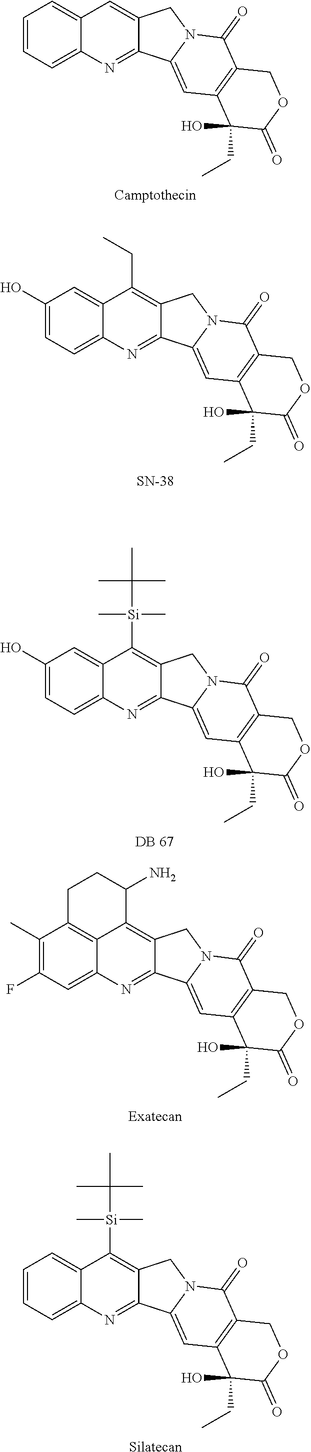

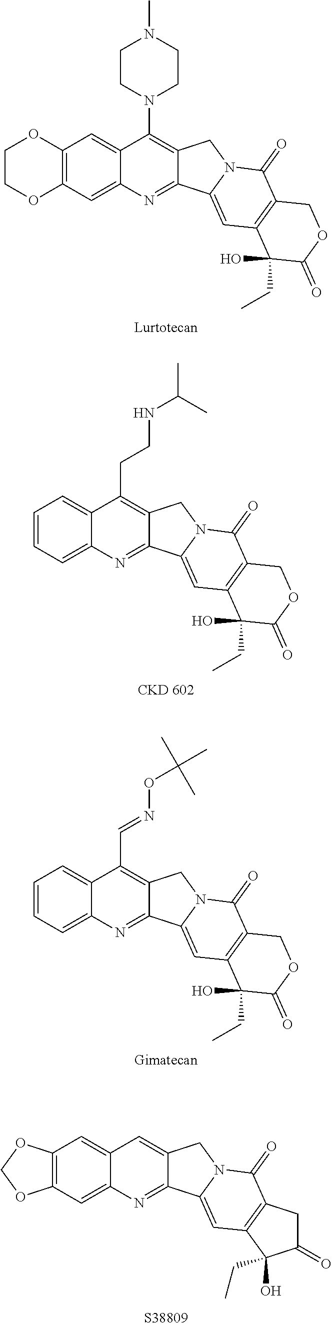

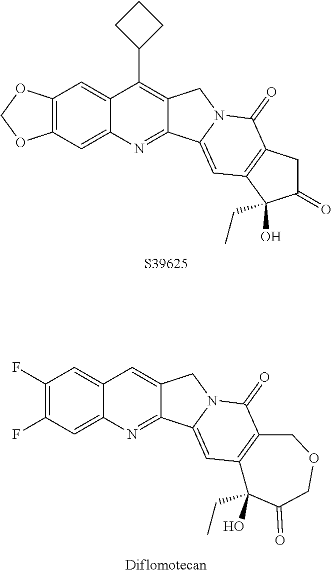 [SMILES: CC[C@@]1(O)C(=O)Cc2c1cc1n(c2=O)Cc2c-1nc1cc3c(cc1c2C1CCC1)OCO3 ; CC[C@@]1(O)C(=O)COCc2c1cc1n(c2=O)Cc2cc3cc(F)c(F)cc3nc2-1]

Several anthracycline derivatives have been produced and have found use in the clinic for the treatment of leukemias, Hodgkin's lymphoma, as well as cancers of the bladder, breast, stomach, lung, ovaries, thyroid, and soft tissue sarcoma. Such anthracycline derivatives include daunorubicin (also known as Daunomycin or daunomycin cerubidine), doxorubicin (also known as DOX, hydroxydaunorubicin, or adriamycin), epirubicin (also known as Ellence or Pharmorubicin), idarubicin (also known as 4-demethoxydaunorubicin, Zavedos, or Idamycin), and valrubicin (also known as N-trifluoroacetyladriamycin-14-valerate or Valstar). Anthracyclines are typically prepared as an ammonium salt (e.g. hydrochloride salt) to improve water solubility and allow for ease of administration.

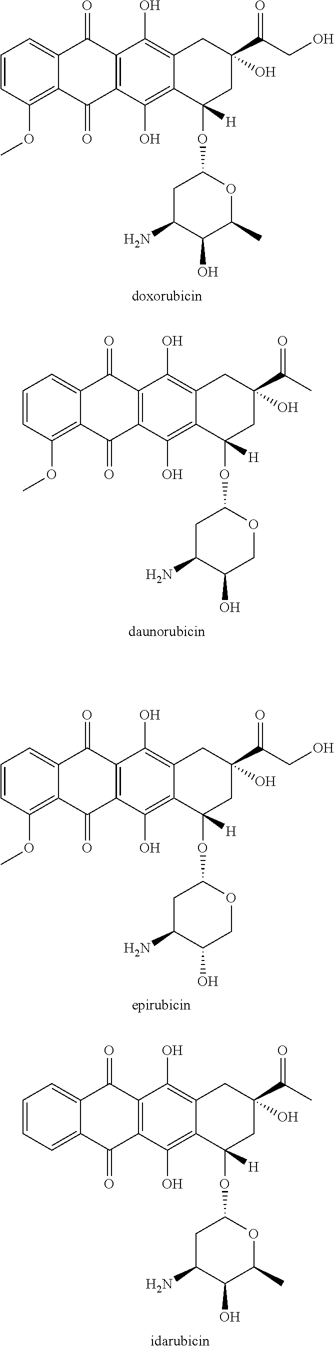 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; COc1cccc2c1C(=O)c1c(O)c3c(c(O)c1C2=O)C[C@@](O)(C(C)=O)C[C@@H]3O[C@H]1C[C@H](N)[C@H](O)CO1 ; COc1cccc2c1C(=O)c1c(O)c3c(c(O)c1C2=O)C[C@@](O)(C(=O)CO)C[C@@H]3O[C@H]1C[C@H](N)[C@@H](O)CO1 ; CC(=O)[C@]1(O)Cc2c(O)c3c(c(O)c2[C@@H](O[C@H]2C[C@H](N)[C@H](O)[C@H](C)O2)C1)C(=O)c1ccccc1C3=O]

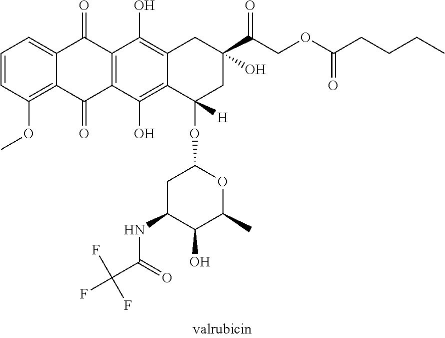 [SMILES: CCCCC(=O)OCC(=O)[C@]1(O)Cc2c(O)c3c(c(O)c2[C@@H](O[C@H]2C[C@H](NC(=O)C(F)(F)F)[C@H](O)[C@H](C)O2)C1)C(=O)c1c(OC)cccc1C3=O]

Aminopterin is well known in the literature and is an analog of folic acid that is an antineoplastic agent. Aminopterin works as an enzyme inhibitor by competing for the folate binding site of the enzyme dihydofolate reductase. The structure of aminopterin is shown below.

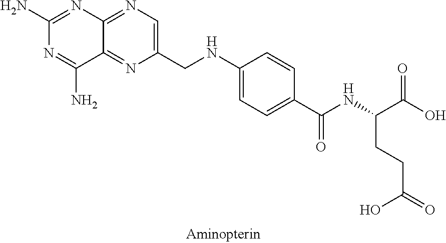 [SMILES: Nc1nc(N)c2nc(CNc3ccc(C(=O)N[C@@H](CCC(=O)O)C(=O)O)cc3)cnc2n1]

Platinum based therapeutics are well known in the literature. Platinum therapeutics are widely used in oncology and act to crosslink DNA which results in cell death (apoptosis). Carboplatin, picoplatin, cisplatin, and oxaliplatin are exemplary platinum therapeutics and the structures are shown below.

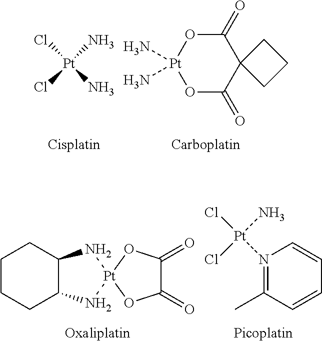

Molecularly targeted therapeutics are well known in the literature. Molecularly targeted therapies are widely used in oncology and act to inhibit specific enzyme activity or to block certain cellular receptors. Tyrosine kinase inhibitors are one subclass of molecularly targeted therapeutics. Other classes of molecularly targeted therapeutics include, but are not limited to, proteasome inhibitors, Janus kinase inhibitors, ALK inhibitors, Bcl-2 inhibitors, PARP inhibitors, PI3K inhibitors, Braf inhibitors, MEK inhibitors, SMAC mimetics, and CDK inhibitors. LY2835219, palociclib, selumetinib, MEK162, trametinib, alisertib, birinapant, LCL161, AT-406, BBI608, KP46, everolimus, and XL147 are exemplary molecularly targeted therapeutics and the structures are shown below.

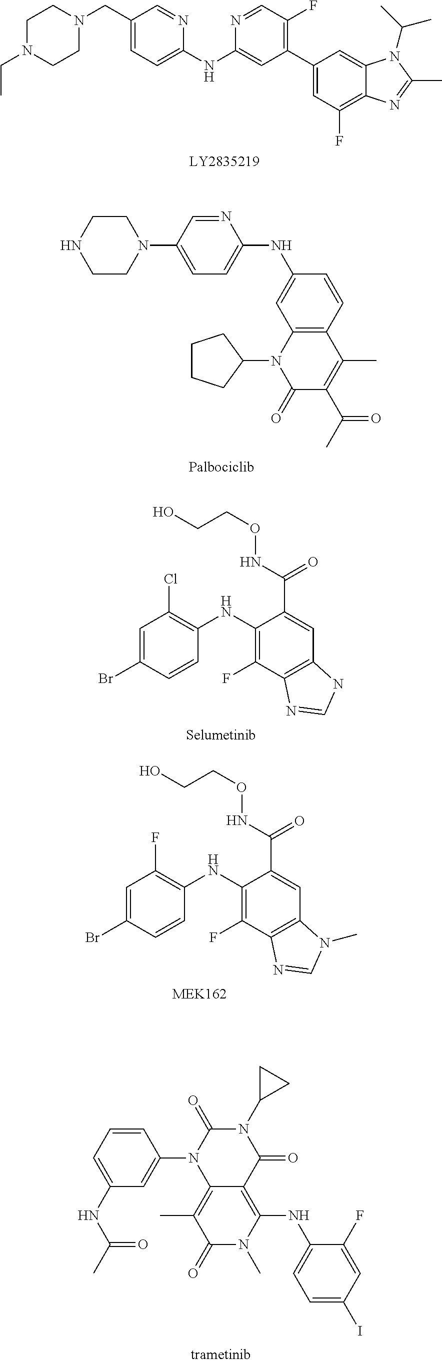 [SMILES: CCN1CCN(Cc2ccc(Nc3cc(-c4cc(F)c5nc(C)n(C(C)C)c5c4)c(F)cn3)nc2)CC1 ; CC(=O)c1c(C)c2ccc(Nc3ccc(N4CCNCC4)cn3)cc2n(C2CCCC2)c1=O ; O=C(NOCCO)c1cc2[nH]cnc2c(F)c1Nc1ccc(Br)cc1Cl ; Cn1cnc2c(F)c(Nc3ccc(Br)cc3F)c(C(=O)NOCCO)cc21 ; CC(=O)Nc1cccc(-n2c(=O)n(C3CC3)c(=O)c3c(Nc4ccc(I)cc4F)n(C)c(=O)c(C)c32)c1]

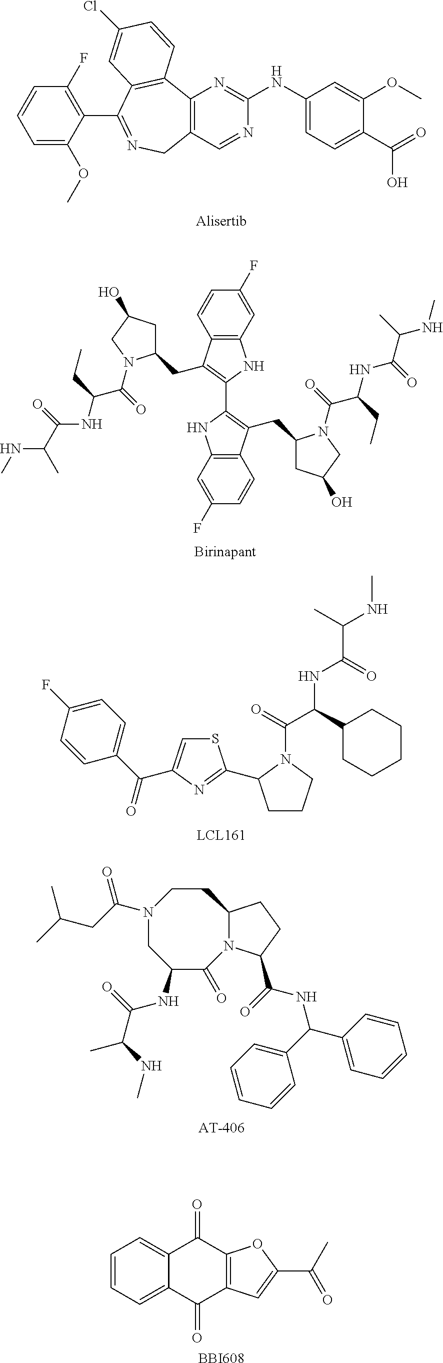

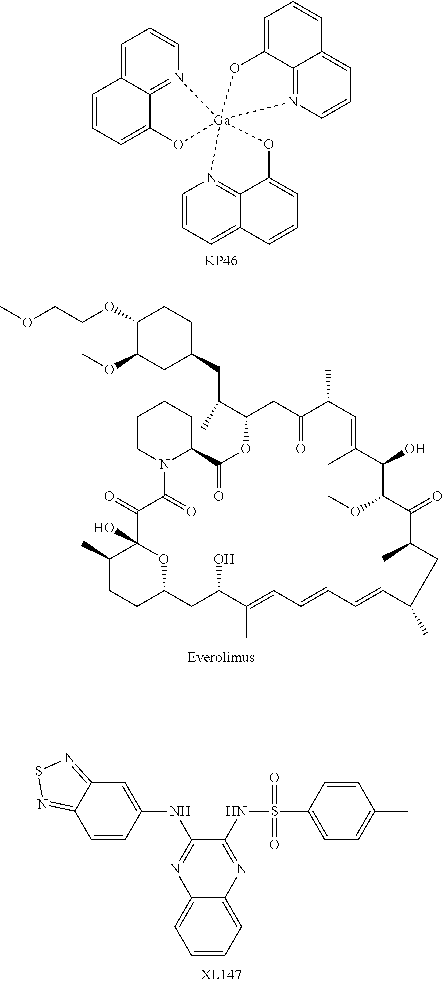

Additional molecularly targeted therapeutics are also in development. Examples include E7016, XL765, TG101348, E7820, eribulin, INK 128, TAK-385, MLN2480, TAK733, MLN-4924, motesanib, ixazomib, TAK-700, dacomitinib, and sunitinib. The structures of each are shown below.

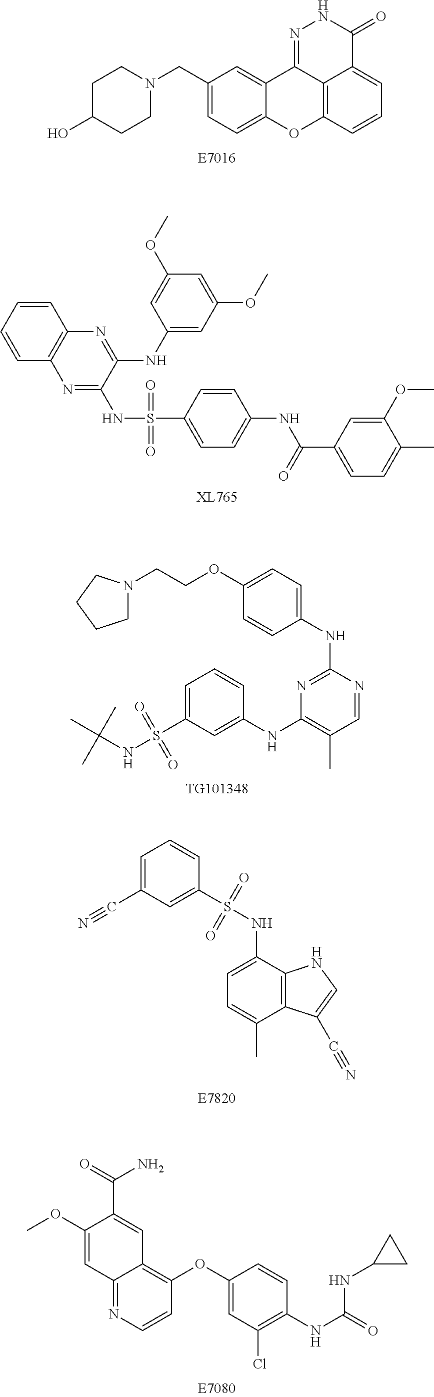

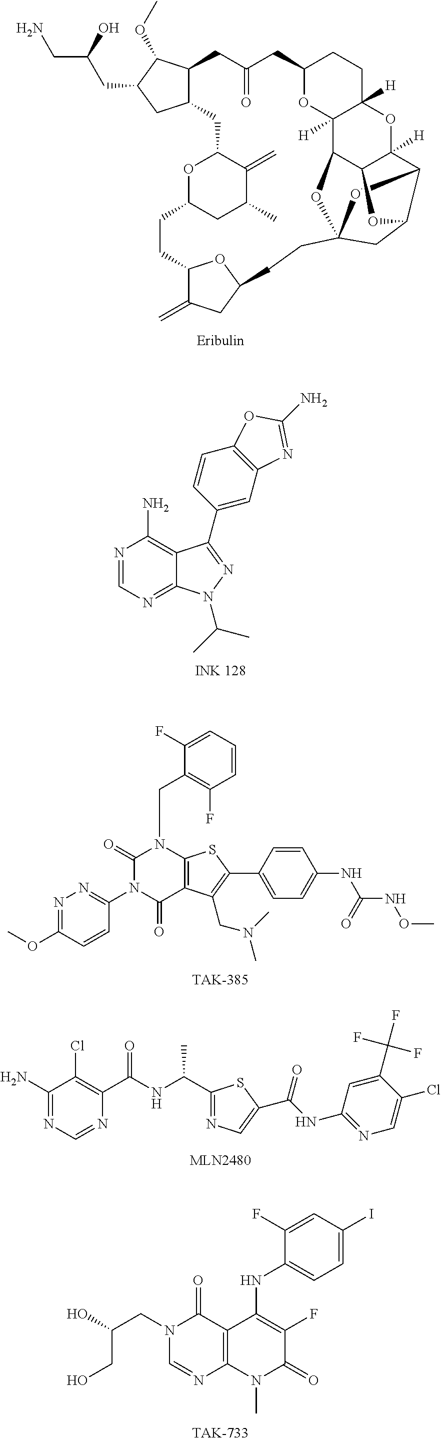

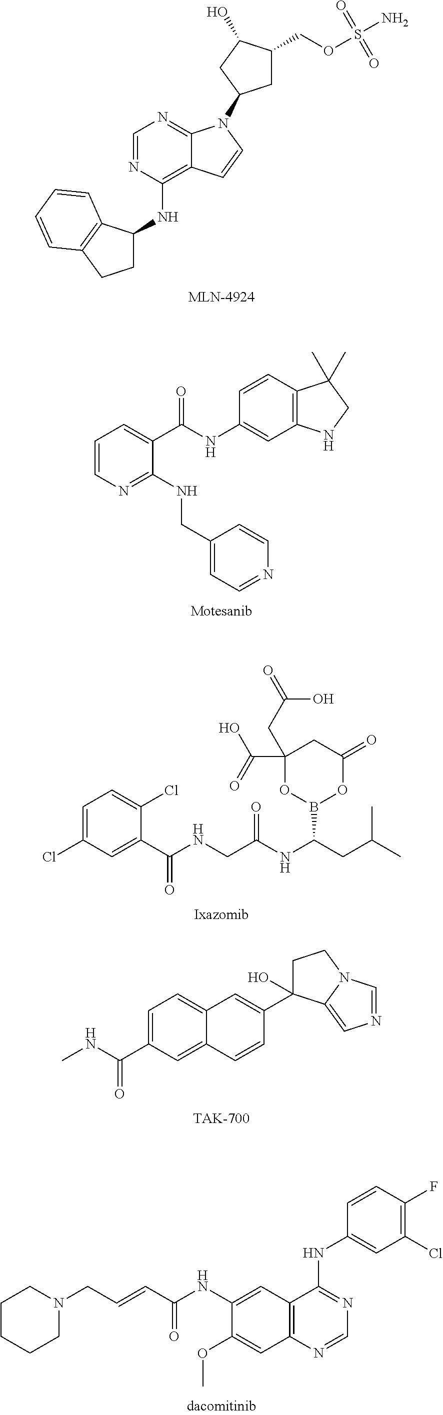

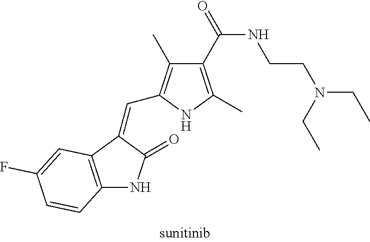 [SMILES: CCN(CC)CCNC(=O)c1c(C)[nH]c(/C=C2\C(=O)Nc3ccc(F)cc32)c1C]

Further examples of molecularly targeted therapeutics include crizotinib, axitinib, PF 03084014, PD 0325901, PF 05212384, PF 04449913, ridaforlimus, MK-1775, MK-2206, GSK2636771, GSK525762, eltrombopag, dabrefenib, and foretinib. The structures of each are shown below.

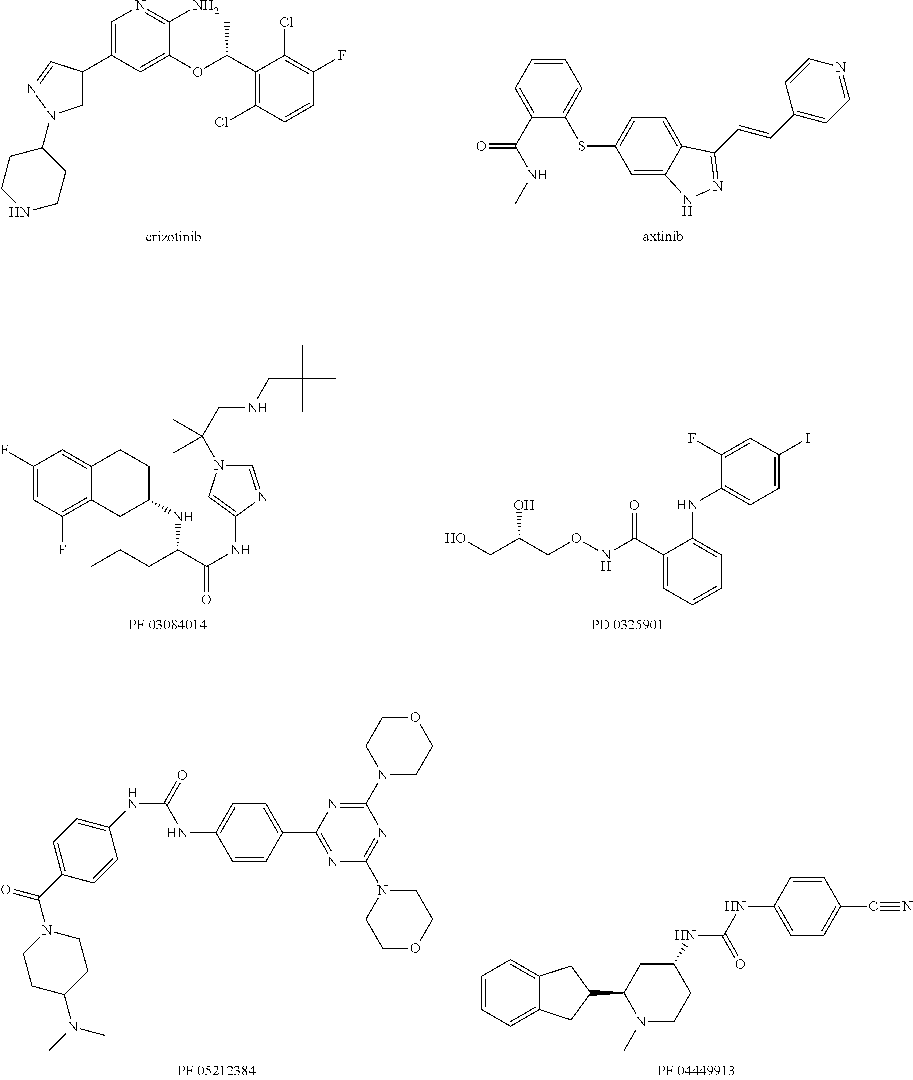 [SMILES: C[C@@H](Oc1cc(C2C=NN(C3CCNCC3)C2)cnc1N)c1c(Cl)ccc(F)c1Cl ; CNC(=O)c1ccccc1Sc1ccc2c(/C=C/c3ccncc3)n[nH]c2c1 ; CCC[C@H](N[C@H]1CCc2cc(F)cc(F)c2C1)C(=O)Nc1cn(C(C)(C)CNCC(C)(C)C)cn1 ; O=C(NOC[C@@H](O)CO)c1ccccc1Nc1ccc(I)cc1F ; CN(C)C1CCN(C(=O)c2ccc(NC(=O)Nc3ccc(-c4nc(N5CCOCC5)nc(N5CCOCC5)n4)cc3)cc2)CC1 ; CN1CC[C@@H](NC(=O)Nc2ccc(C#N)cc2)C[C@@H]1C1Cc2ccccc2C1]

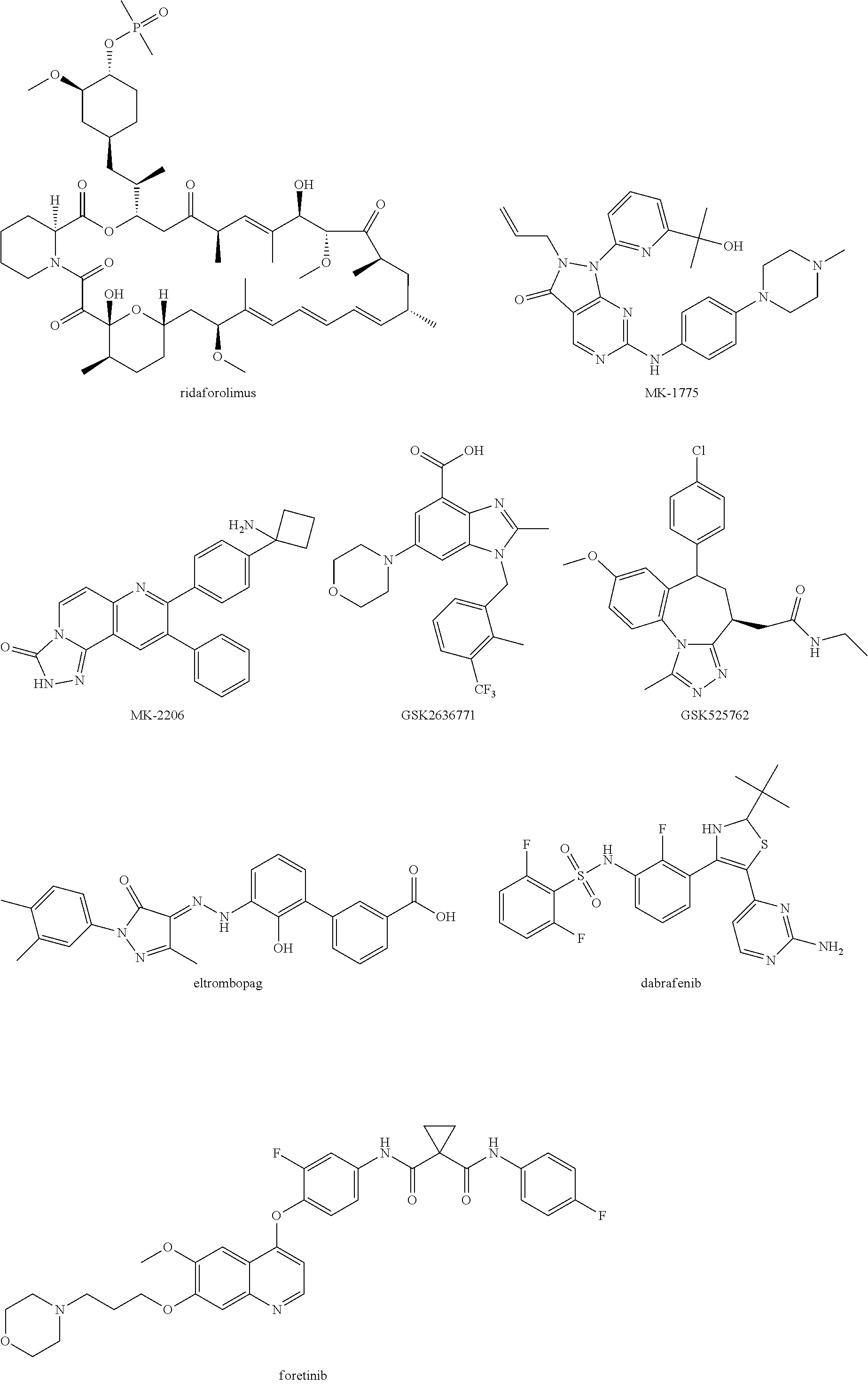

Yet further examples of molecularly targeted therapeutics include lapatinib, pazopanib, CH5132799, RO4987655, RG7338, A0379, erlotinib, pictilisib, GDC-0032, venurafenib, GDC-0980, GDC-0068, arry-520, pasireotide, dovitinib, and cobmetinib. The structures of each are shown below.

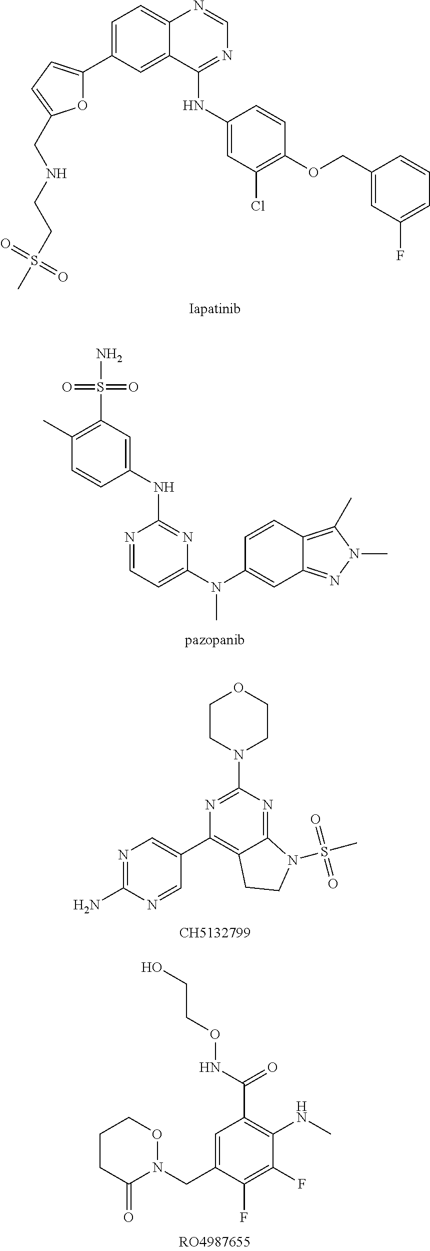

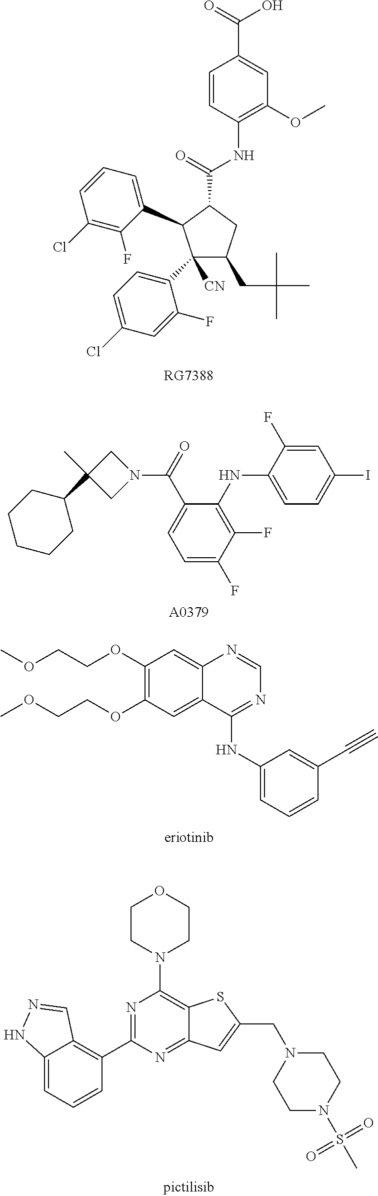

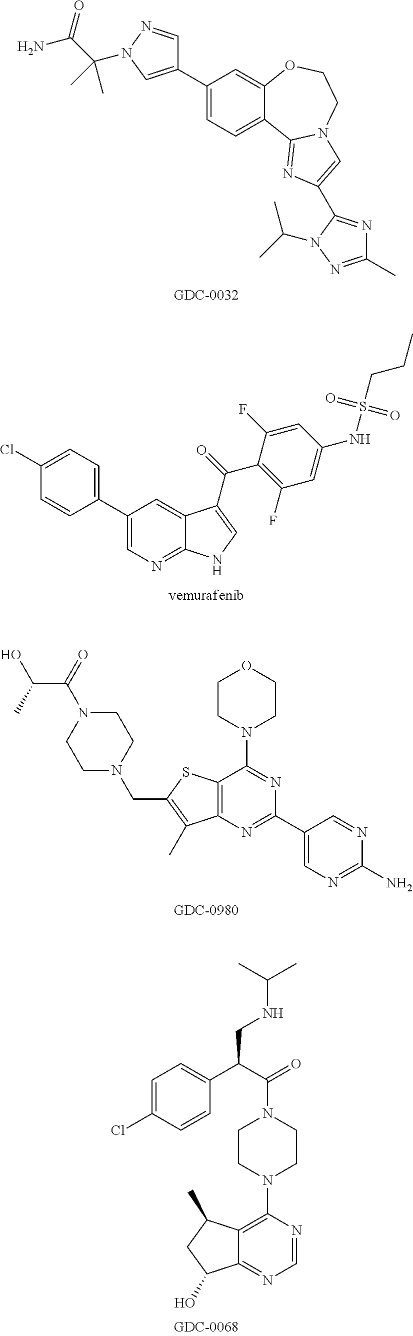

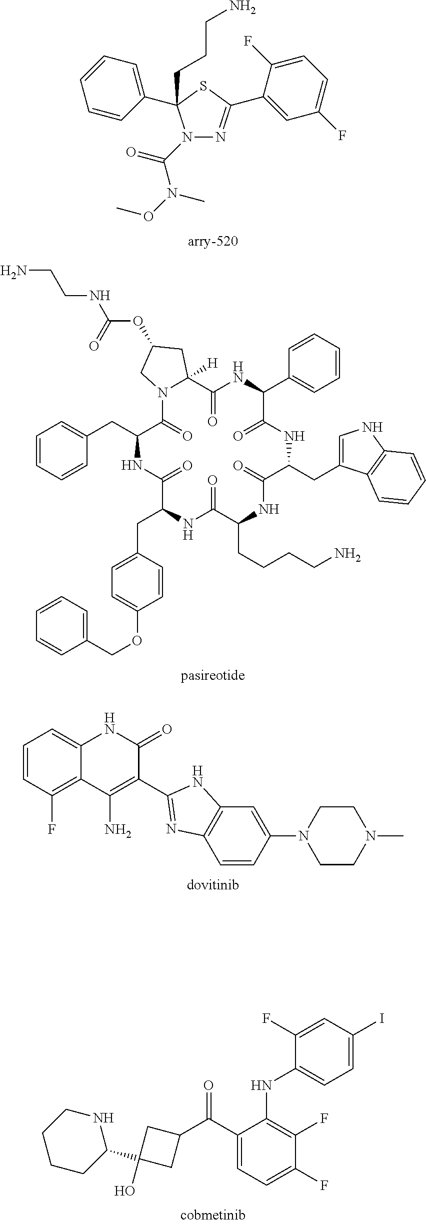 [SMILES: CON(C)C(=O)N1N=C(c2cc(F)ccc2F)S[C@@]1(CCCN)c1ccccc1 ; NCCCC[C@@H]1NC(=O)[C@@H](Cc2c[nH]c3ccccc23)NC(=O)[C@H](c2ccccc2)NC(=O)[C@@H]2C[C@@H](OC(=O)NCCN)CN2C(=O)[C@H](Cc2ccccc2)NC(=O)[C@H](Cc2ccc(OCc3ccccc3)cc2)NC1=O ; CN1CCN(c2ccc3nc(-c4c(N)c5c(F)cccc5[nH]c4=O)[nH]c3c2)CC1 ; O=C(c1ccc(F)c(F)c1Nc1ccc(I)cc1F)C1CC(O)([C@@H]2CCCCN2)C1]

Additional examples of molecularly targeted therapeutics include buparlisib, AVL-292, romidepsin, arry-797, lenalidomide, thalidomide, apremilast, AMG-900, AMG208, rucaparib, NVP-BEZ 235, AUY922, LDE225, and midostaurin. The structures of each are shown below.

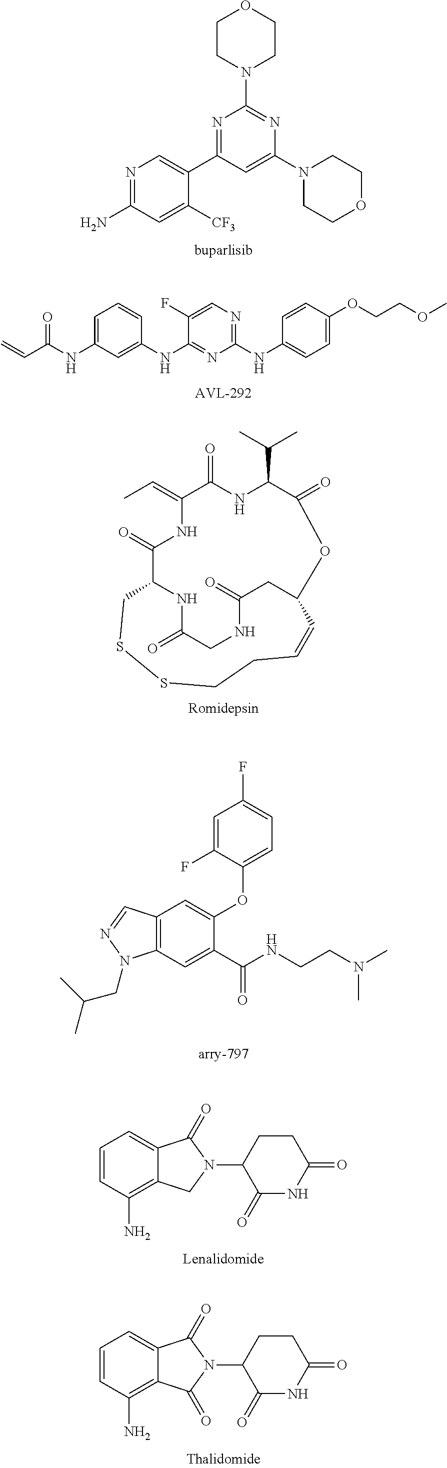

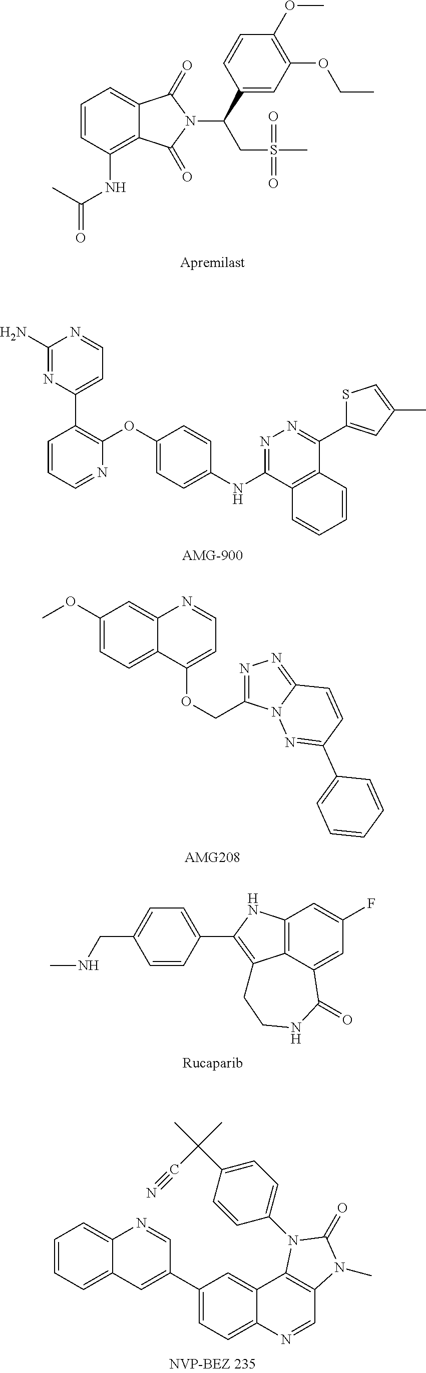

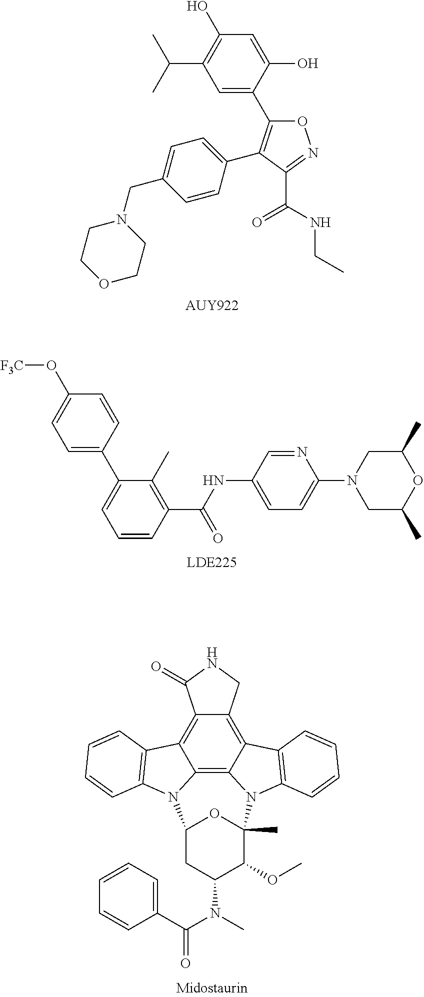 [SMILES: CCNC(=O)c1noc(-c2cc(C(C)C)c(O)cc2O)c1-c1ccc(CN2CCOCC2)cc1 ; "AUY922" ; Cc1c(C(=O)Nc2ccc(N3C[C@@H](C)O[C@@H](C)C3)nc2)cccc1-c1ccc(OC(F)(F)F)cc1 ; CO[C@@H]1[C@H](N(C)C(=O)c2ccccc2)C[C@H]2O[C@]1(C)n1c3ccccc3c3c4c(c5c6ccccc6n2c5c31)C(=O)NC4]

In certain embodiments, the present invention provides a drug-loaded, iron stabilized micelle that provides contrast in magnetic resonance imaging, as described herein, wherein the drug is a tyrosine kinase inhibitor.

In certain embodiments, the present invention provides a drug-loaded, iron stabilized micelle that provides contrast in magnetic resonance imaging, as described herein, wherein the drug is a molecularly targeted therapeutic.

In certain embodiments, the present invention provides a drug-loaded, iron stabilized micelle that provides contrast in magnetic resonance imaging, as described herein, wherein the drug is LY2835219.

In certain embodiments, the present invention provides a drug-loaded, iron stabilized micelle that provides contrast in magnetic resonance imaging, as described herein, wherein the drug is palbociclib.

In certain embodiments, the present invention provides a drug-loaded, iron stabilized micelle that provides contrast in magnetic resonance imaging, as described herein, wherein the drug is selumetinib.

In certain embodiments, the present invention provides a drug-loaded, iron stabilized micelle that provides contrast in magnetic resonance imaging, as described herein, wherein the drug is MEK162.

In certain embodiments, the present invention provides a drug-loaded, iron stabilized micelle that provides contrast in magnetic resonance imaging, as described herein, wherein the drug is trametinib.

In certain embodiments, the present invention provides a drug-loaded, iron stabilized micelle that provides contrast in magnetic resonance imaging, as described herein, wherein the drug is alisertib.

In certain embodiments, the present invention provides a drug-loaded, iron stabilized micelle that provides contrast in magnetic resonance imaging, as described herein, wherein the drug is birinapant.

In certain embodiments, the present invention provides a drug-loaded, iron stabilized micelle that provides contrast in magnetic resonance imaging, as described herein, wherein the drug is LCL161.

In certain embodiments, the present invention provides a drug-loaded, iron stabilized micelle that provides contrast in magnetic resonance imaging, as described herein, wherein the drug is AT-406.

In certain embodiments, the present invention provides a drug-loaded, iron stabilized micelle that provides contrast in magnetic resonance imaging, as described herein, wherein the drug is BB1608.

In certain embodiments, the present invention provides a drug-loaded, iron stabilized micelle that provides contrast in magnetic resonance imaging, as described herein, wherein the drug is KP46 [tris(8-quinolinolato)gallium(III)].

In certain embodiments, the present invention provides a drug-loaded, iron stabilized micelle that provides contrast in magnetic resonance imaging, as described herein, wherein the drug is everolimus.

In certain embodiments, the present invention provides a drug-loaded, iron stabilized micelle that provides contrast in magnetic resonance imaging, as described herein, wherein the drug is XL 147.

In certain embodiments, the present invention provides a drug-loaded, iron stabilized micelle that provides contrast in magnetic resonance imaging, as described herein, wherein the drug is E7016.

In certain embodiments, the present invention provides a drug-loaded, iron stabilized micelle that provides contrast in magnetic resonance imaging, as described herein, wherein the drug is XL765.

In certain embodiments, the present invention provides a drug-loaded, iron stabilized micelle that provides contrast in magnetic resonance imaging, as described herein, wherein the drug is TG101348.

In certain embodiments, the present invention provides a drug-loaded, iron stabilized micelle that provides contrast in magnetic resonance imaging, as described herein, wherein the drug is E7820.

In certain embodiments, the present invention provides a drug-loaded, iron stabilized micelle that provides contrast in magnetic resonance imaging, as described herein, wherein the drug is eribulin.

In certain embodiments, the present invention provides a drug-loaded, iron stabilized micelle that provides contrast in magnetic resonance imaging, as described herein, wherein the drug is INK 128.

In certain embodiments, the present invention provides a drug-loaded, iron stabilized micelle that provides contrast in magnetic resonance imaging, as described herein, wherein the drug is TAK-385.

In certain embodiments, the present invention provides a drug-loaded, iron stabilized micelle that provides contrast in magnetic resonance imaging, as described herein, wherein the drug is MLN2480.

In certain embodiments, the present invention provides a drug-loaded, iron stabilized micelle that provides contrast in magnetic resonance imaging, as described herein, wherein the drug is TAK733.

In certain embodiments, the present invention provides a drug-loaded, iron stabilized micelle that provides contrast in magnetic resonance imaging, as described herein, wherein the drug is MLN-4924.

In certain embodiments, the present invention provides a drug-loaded, iron stabilized micelle that provides contrast in magnetic resonance imaging, as described herein, wherein the drug is motesanib.

In certain embodiments, the present invention provides a drug-loaded, iron stabilized micelle that provides contrast in magnetic resonance imaging, as described herein, wherein the drug is ixazomib.

In certain embodiments, the present invention provides a drug-loaded, iron stabilized micelle that provides contrast in magnetic resonance imaging, as described herein, wherein the drug is TAK-700.

In certain embodiments, the present invention provides a drug-loaded, iron stabilized micelle that provides contrast in magnetic resonance imaging, as described herein, wherein the drug is dacomitinib.

In certain embodiments, the present invention provides a drug-loaded, iron stabilized micelle that provides contrast in magnetic resonance imaging, as described herein, wherein the drug is sunitinib.

In certain embodiments, the present invention provides a drug-loaded, iron stabilized micelle that provides contrast in magnetic resonance imaging, as described herein, wherein the drug is crizotinib.

In certain embodiments, the present invention provides a drug-loaded, iron stabilized micelle that provides contrast in magnetic resonance imaging, as described herein, wherein the drug is axitnib.

In certain embodiments, the present invention provides a drug-loaded, iron stabilized micelle that provides contrast in magnetic resonance imaging, as described herein, wherein the drug is PF 03084014.

In certain embodiments, the present invention provides a drug-loaded, iron stabilized micelle that provides contrast in magnetic resonance imaging, as described herein, wherein the drug is PD 0325901.

In certain embodiments, the present invention provides a drug-loaded, iron stabilized micelle that provides contrast in magnetic resonance imaging, as described herein, wherein the drug is PF05212384.

In certain embodiments, the present invention provides a drug-loaded, iron stabilized micelle that provides contrast in magnetic resonance imaging, as described herein, wherein the drug is PF 04449913.

In certain embodiments, the present invention provides a drug-loaded, iron stabilized micelle that provides contrast in magnetic resonance imaging, as described herein, wherein the drug is ridaforlimus.

In certain embodiments, the present invention provides a drug-loaded, iron stabilized micelle that provides contrast in magnetic resonance imaging, as described herein, wherein the drug is MK-1775.

In certain embodiments, the present invention provides a drug-loaded, iron stabilized micelle that provides contrast in magnetic resonance imaging, as described herein, wherein the drug is MK-2206.

In certain embodiments, the present invention provides a drug-loaded, iron stabilized micelle that provides contrast in magnetic resonance imaging, as described herein, wherein the drug is GSK2636771.

In certain embodiments, the present invention provides a drug-loaded, iron stabilized micelle that provides contrast in magnetic resonance imaging, as described herein, wherein the drug is GSK525762.

In certain embodiments, the present invention provides a drug-loaded, iron stabilized micelle that provides contrast in magnetic resonance imaging, as described herein, wherein the drug is eltrombopag.

In certain embodiments, the present invention provides a drug-loaded, iron stabilized micelle that provides contrast in magnetic resonance imaging, as described herein, wherein the drug is dabrefenib.

In certain embodiments, the present invention provides a drug-loaded, iron stabilized micelle that provides contrast in magnetic resonance imaging, as described herein, wherein the drug is foretinib.

In certain embodiments, the present invention provides a drug-loaded, iron stabilized micelle that provides contrast in magnetic resonance imaging, as described herein, wherein the drug is lapatinib.

In certain embodiments, the present invention provides a drug-loaded, iron stabilized micelle that provides contrast in magnetic resonance imaging, as described herein, wherein the drug is pazopanib.

In certain embodiments, the present invention provides a drug-loaded, iron stabilized micelle that provides contrast in magnetic resonance imaging, as described herein, wherein the drug is CH5132799.

In certain embodiments, the present invention provides a drug-loaded, iron stabilized micelle that provides contrast in magnetic resonance imaging, as described herein, wherein the drug is RO4987655.

In certain embodiments, the present invention provides a drug-loaded, iron stabilized micelle that provides contrast in magnetic resonance imaging, as described herein, wherein the drug is RG7338.

In certain embodiments, the present invention provides a drug-loaded, iron stabilized micelle that provides contrast in magnetic resonance imaging, as described herein, wherein the drug is A0379.

In certain embodiments, the present invention provides a drug-loaded, iron stabilized micelle that provides contrast in magnetic resonance imaging, as described herein, wherein the drug is erlotinib.

In certain embodiments, the present invention provides a drug-loaded, iron stabilized micelle that provides contrast in magnetic resonance imaging, as described herein, wherein the drug is pictilisib.

In certain embodiments, the present invention provides a drug-loaded, iron stabilized micelle that provides contrast in magnetic resonance imaging, as described herein, wherein the drug is GDC-0032.

In certain embodiments, the present invention provides a drug-loaded, iron stabilized micelle that provides contrast in magnetic resonance imaging, as described herein, wherein the drug is venurafenib.

In certain embodiments, the present invention provides a drug-loaded, iron stabilized micelle that provides contrast in magnetic resonance imaging, as described herein, wherein the drug is GDC-0980.

In certain embodiments, the present invention provides a drug-loaded, iron stabilized micelle that provides contrast in magnetic resonance imaging, as described herein, wherein the drug is GDC-0068.

In certain embodiments, the present invention provides a drug-loaded, iron stabilized micelle that provides contrast in magnetic resonance imaging, as described herein, wherein the drug is arry-520.

In certain embodiments, the present invention provides a drug-loaded, iron stabilized micelle that provides contrast in magnetic resonance imaging, as described herein, wherein the drug is pasireotide.

In certain embodiments, the present invention provides a drug-loaded, iron stabilized micelle that provides contrast in magnetic resonance imaging, as described herein, wherein the drug is dovitinib.

In certain embodiments, the present invention provides a drug-loaded, iron stabilized micelle that provides contrast in magnetic resonance imaging, as described herein, wherein the drug is cobmetinib.

In certain embodiments, the present invention provides a drug-loaded, iron stabilized micelle that provides contrast in magnetic resonance imaging, as described herein, wherein the drug is buparlisib.

In certain embodiments, the present invention provides a drug-loaded, iron stabilized micelle that provides contrast in magnetic resonance imaging, as described herein, wherein the drug is AVL-292.

In certain embodiments, the present invention provides a drug-loaded, iron stabilized micelle that provides contrast in magnetic resonance imaging, as described herein, wherein the drug is romidepsin.

In certain embodiments, the present invention provides a drug-loaded, iron stabilized micelle that provides contrast in magnetic resonance imaging, as described herein, wherein the drug is arry-797.

In certain embodiments, the present invention provides a drug-loaded, iron stabilized micelle that provides contrast in magnetic resonance imaging, as described herein, wherein the drug is lenalidomide.

In certain embodiments, the present invention provides a drug-loaded, iron stabilized micelle that provides contrast in magnetic resonance imaging, as described herein, wherein the drug is thalidomide.

In certain embodiments, the present invention provides a drug-loaded, iron stabilized micelle that provides contrast in magnetic resonance imaging, as described herein, wherein the drug is apremilast.

In certain embodiments, the present invention provides a drug-loaded, iron stabilized micelle that provides contrast in magnetic resonance imaging, as described herein, wherein the drug is AMG-900.

In certain embodiments, the present invention provides a drug-loaded, iron stabilized micelle that provides contrast in magnetic resonance imaging, as described herein, wherein the drug is AMG208.

In certain embodiments, the present invention provides a drug-loaded, iron stabilized micelle that provides contrast in magnetic resonance imaging, as described herein, wherein the drug is rucaparib.

In certain embodiments, the present invention provides a drug-loaded, iron stabilized micelle that provides contrast in magnetic resonance imaging, as described herein, wherein the drug is NVP-BEZ 235.

In certain embodiments, the present invention provides a drug-loaded, iron stabilized micelle that provides contrast in magnetic resonance imaging, as described herein, wherein the drug is AUY922.

In certain embodiments, the present invention provides a drug-loaded, iron stabilized micelle that provides contrast in magnetic resonance imaging, as described herein, wherein the drug is LDE225.

In certain embodiments, the present invention provides a drug-loaded, iron stabilized micelle that provides contrast in magnetic resonance imaging, as described herein, wherein the drug is midostaurin.

Small molecule drugs suitable for loading into micelles of the present invention are well known in the art. In certain embodiments, the present invention provides a drug-loaded micelle as described herein, wherein the drug is a hydrophobic drug selected from analgesics, anti-inflammatory agents, HDAC inhibitors, mitotic inhibitors, microtubule stabilizers, DNA intercalators, topoisomerase inhibitors, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof.

In other embodiments, the hydrophobic drug is selected from one or more analgesics, anti-bacterial agents, anti-viral agents, anti-inflammatory agents, anti-depressants, anti-diabetics, anti-epileptics, anti-hypertensive agents, anti-migraine agents, immunosuppressants, anxiolytic agents, sedatives, hypnotics, neuroleptics, β-blockers, gastro-intestinal agents, lipid regulating agents, anti-anginal agents, Cox-2 inhibitors, leukotriene inhibitors, macrolides, muscle relaxants, opioid analgesics, protease inhibitors, sex hormones, cognition enhancers, anti-urinary incontinence agents, and mixtures thereof.

According to one aspect, the present invention provides a micelle, as described herein, loaded with a hydrophobic drug selected from any one or more of a Exemestance (aromasin), Camptosar (irinotecan), Ellence (epirubicin), Femara (Letrozole), Gleevac (imatinib mesylate), Lentaron (formestane), Cytadren/Orimeten (aminoglutethimide), Temodar, Proscar (finasteride), Viadur (leuprolide), Nexavar (Sorafenib), Kytril (Granisetron), Taxotere (Docetaxel), Taxol (paclitaxel), Kytril (Granisetron), Vesanoid (tretinoin) (retin A), XELODA (Capecitabine), Arimidex (Anastrozole), Casodex/Cosudex (Bicalutamide), Faslodex (Fulvestrant), Iressa (Gefitinib), Nolvadex, Istubal, Valodex (tamoxifen citrate), Tomudex (Raltitrexed), Zoladex (goserelin acetate), Leustatin (Cladribine), Velcade (bortezomib), Mylotarg (gemtuzumab ozogamicin), Alimta (pemetrexed), Gemzar (gemcitabine hydrochloride), Rituxan (rituximab), Revlimid (lenalidomide), Thalomid (thalidomide), Alkeran (melphalan), and derivatives thereof.

### 4. General Methods for Providing Compounds of the Present Invention

The preparation of drug loaded, iron stabilized micelles in accordance with the present invention is accomplished by methods known in the art, including those described in detail in U.S. patent application Ser. No. 13/839,715, filed Mar. 15, 2013, published as 20130296531 on Nov. 7, 2013, the entirety of which is hereby incorporated herein by reference. Additionally methods know in the art include those described in detail in U.S. patent application Ser. No. 12/112,799, filed Feb. 29, 2008, published as 20090092554 on Apr. 9, 2009, the entirety of which is hereby incorporated herein by reference.

Compositions

According to another embodiment, the invention provides a composition comprising a micelle of this invention or a pharmaceutically acceptable derivative thereof and a pharmaceutically acceptable carrier, adjuvant, or vehicle. In certain embodiments, the composition of this invention is formulated for administration to a subject in need of such composition. In other embodiments, the composition of this invention is formulated for oral administration to a subject.

The term “subject”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of acid salts include acetate, adipate, alginate, aspartate, benzoate, benzenesulfonate, bisulfate, butyrate, citrate, camphorate, camphorsulfonate, cyclopentanepropionate, digluconate, dodecyl sulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In certain embodiments, pharmaceutically acceptable compositions of the present invention are enterically coated.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other preservatives, absorption promoters to enhance bioavailability, fluorocarbons, and/or other conventional solubilizing or dispersing agents.

In certain embodiments, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-5,000 mg/kg body weight/day of the drug can be administered to a subject receiving these compositions.

It will be appreciated that dosages typically employed for the encapsulated drug are contemplated by the present invention. In certain embodiments, a subject is administered a drug-loaded micelle of the present invention wherein the dosage of the drug is equivalent to what is typically administered for that drug. In other embodiments, a subject is administered a drug-loaded micelle of the present invention wherein the dosage of the drug is lower than is typically administered for that drug.

It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### EXEMPLIFICATION

In order that the invention described herein may be more fully understood, the following examples are set forth. It will be understood that these examples are for illustrative purposes only and are not to be construed as limiting this invention in any manner.

### Example 1

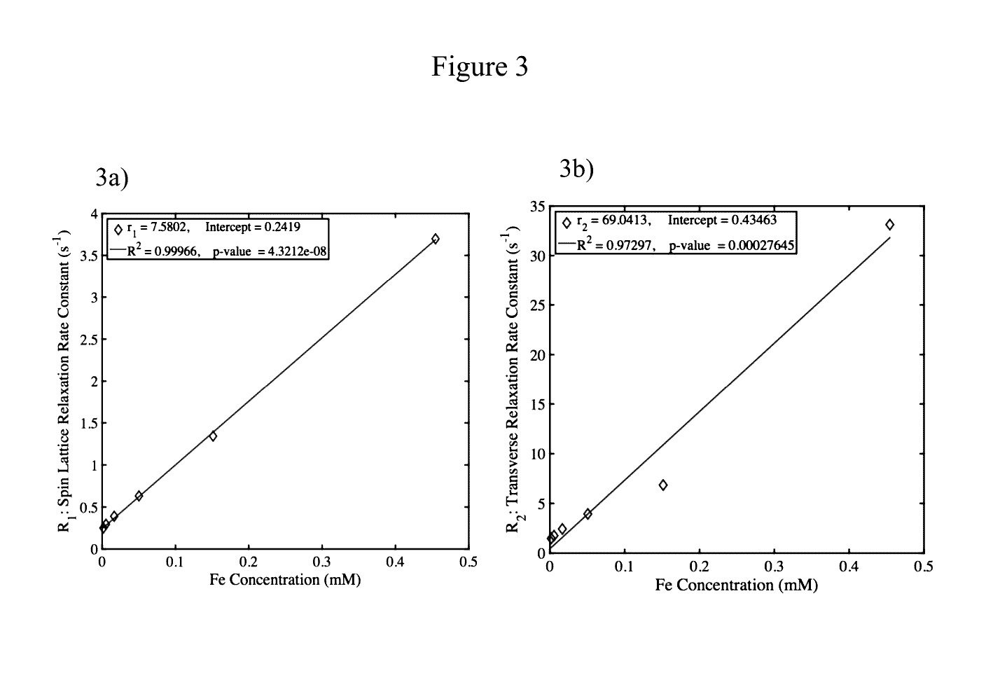

In vitro phantom measurements were performed to determine spin-lattice (r₁) and spin-spin (r₂) relaxivity values. Multiple concentrations of each nanoparticle were prepared at values ranging from 0.002 mM to 0.6 mM and repeated in triplicate in a 96-well plate cut into two. This was placed directly in a 72 mm ID birdcage coil in a horizontal bore magnet at 7 Tesla (Agilent ASR 310) and spin echo images (sems) were acquired at either multiple TR values (progressive saturation) or followed by a train of 180° pulses for collection of multiple spin echoes (mems). These images were collected with a field of view of 8×4 cm² and a matrix size of 256×128. Mean values were obtained from regions of interest within each voxel and used to fit the relaxivity with a nonlinear least squares fit using the Levenberg-Marquardt algorithm. Estimates of the each relaxivity parameter (n=1 for spin lattice or n=2 for spin-spin) were determined by linear regression of the expression rₙ=(Rₙ−R_{n,0})/[Fe]. The results of the phantom measurements for non-drug loaded, iron stabilized micelles are shown in FIG. 2. Spin-lattice relaxivity (r₁) was found to be 10.9 mmol⁻¹s⁻¹. Spin-spin relaxivity (r₂) was found to be 53.5 mmol⁻¹s⁻¹. The results of the phantom measurements for SN-38 loaded, iron stabilized micelles are shown in FIG. 3. Spin-lattice relaxivity (r₁) was found to be 7.6 mmol⁻¹s⁻¹. Spin-spin relaxivity (r₂) was found to be 69.0 mmol⁻¹s⁻¹. The results of the phantom measurements for Epothilone D loaded, iron stabilized micelles are as follows: spin-lattice relaxivity (r₁) was found to be 16.2 mmol⁻¹s⁻¹ and Spin-spin relaxivity (r₂) was found to be 80.1 mmol⁻¹s⁻¹. These results demonstrate that the iron-stabilized micelles of the present invention are suitable magnetic contrast agents independent of the encapsulated molecule. These data further demonstrate that similar relaxivity data is obtained for each iron-stabilized micelle, regardless of the molecule encapsulated in the micelle core, suggesting that superparamagnetic property of the nanoparticle is a function of the iron-stabilized micelle, rather than the therapeutic.

### Example 2

All in vivo imaging experiments were done in a 7T horizontal magnet (ASR 310, Agilent Technologies, Inc.) with 205/120/HDS gradients and 310 mm bore, using a 35-mm Litzcage coil (Doty Scientific). Mice were anesthetized with 2% isoflurane and restrained in a specific holder. Whole body coronal slices were acquired using a multislice spin-echo (SEMS) sequence with TR/TE 315/7.43 ms, 17 slices, 1 mm slice thickness and 2 averages, FOV=80×40 mm 256×128 pixels. Images were acquired before drug injection, and again at multiple intervals post administration to monitor nanoparticle distribution and clearance. Tumors were manually segmented using a Matlab script to calculate mean and standard deviation of each entire tumor as well as tumor histograms. Regions of Interest (ROIs) in kidneys, liver, muscle were also drawn manually with the same Matlab script to monitor drug clearance.

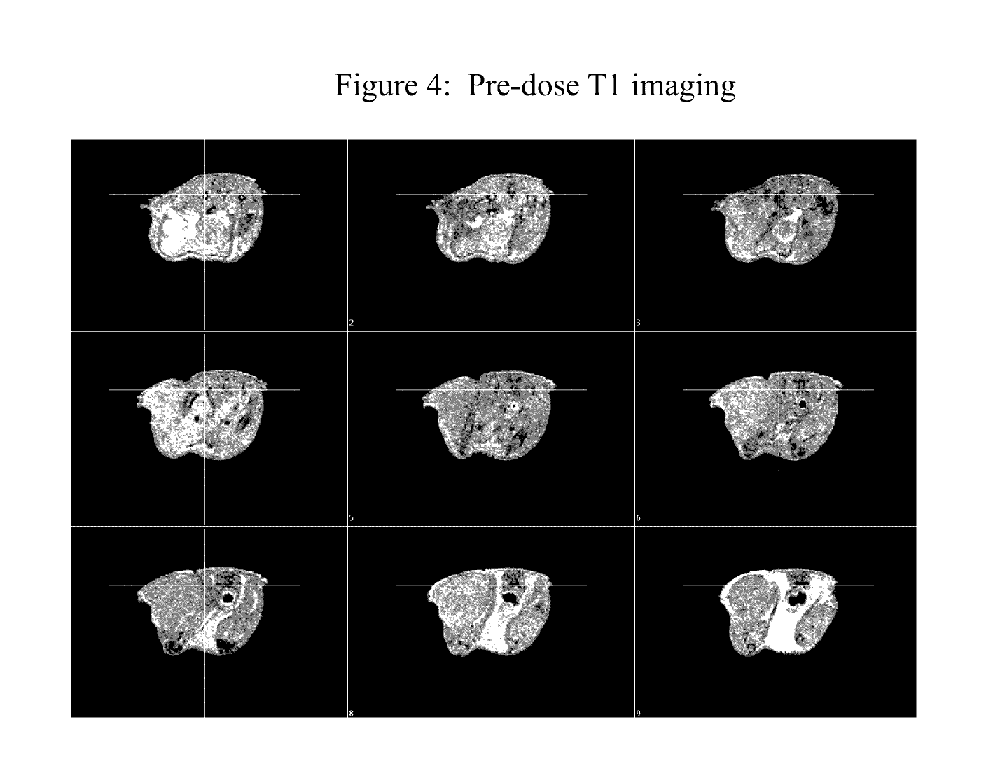

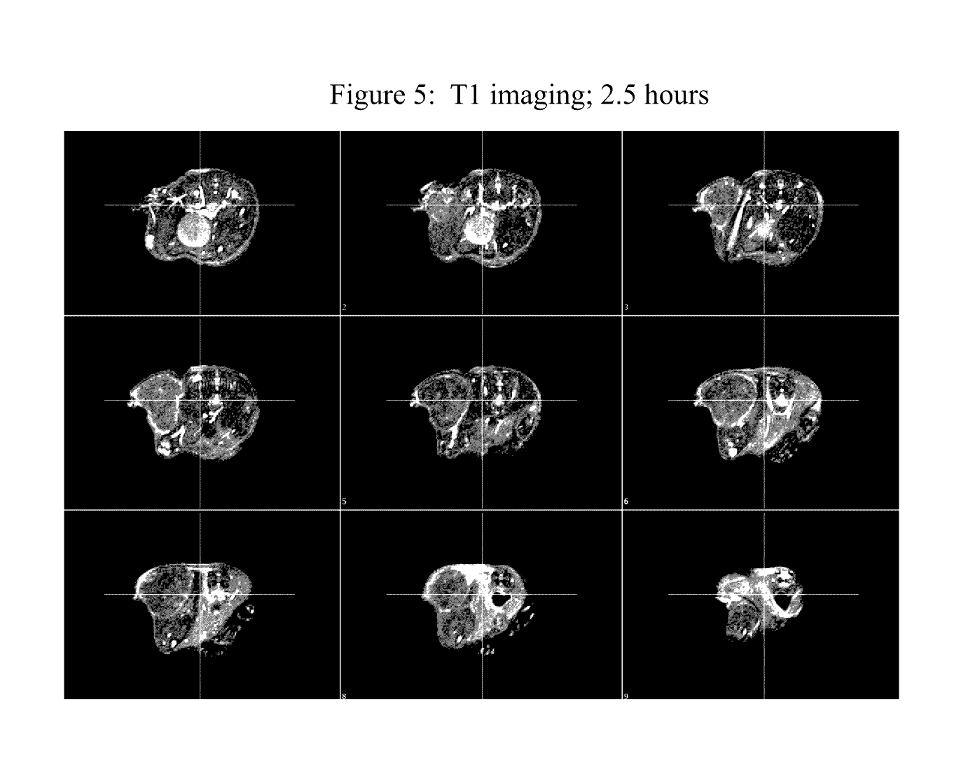

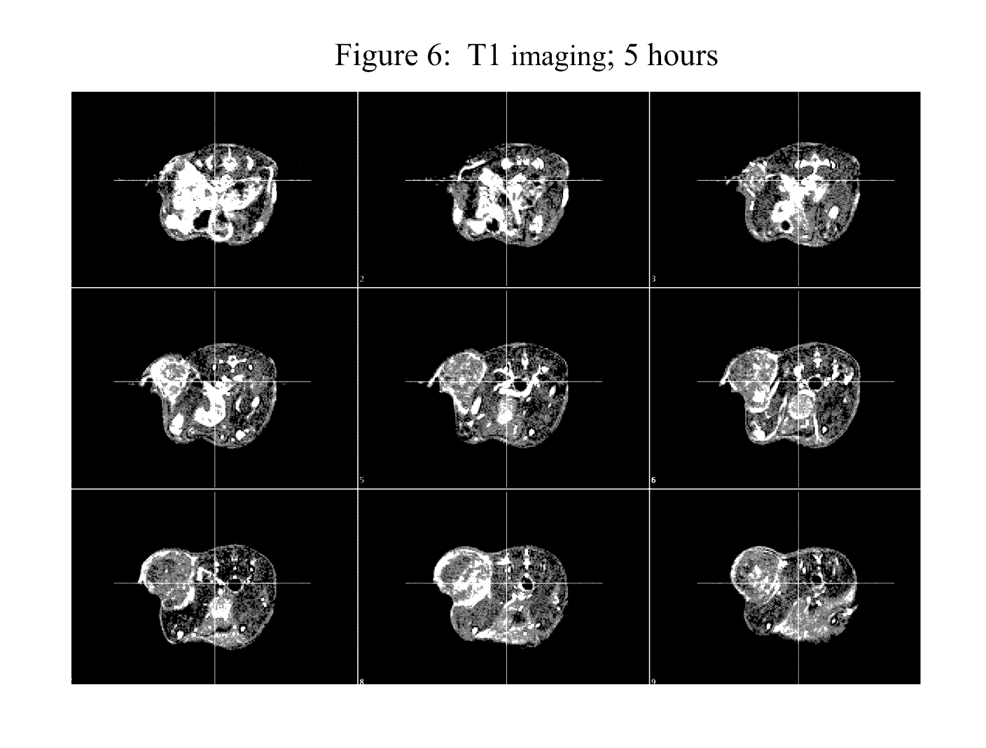

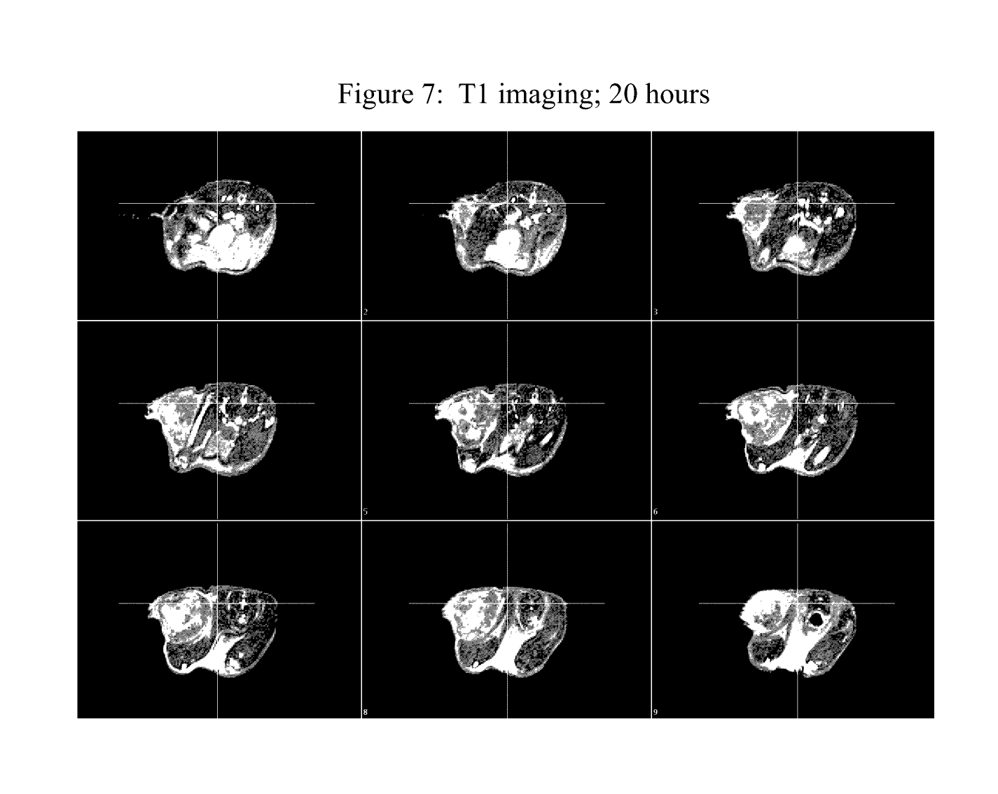

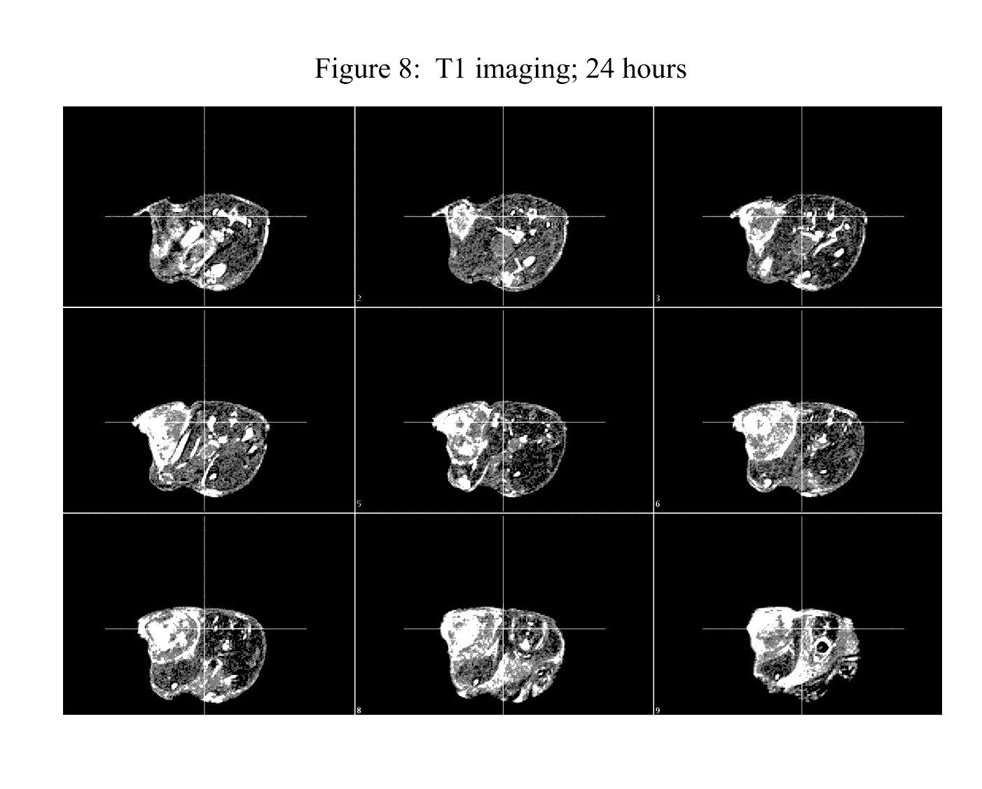

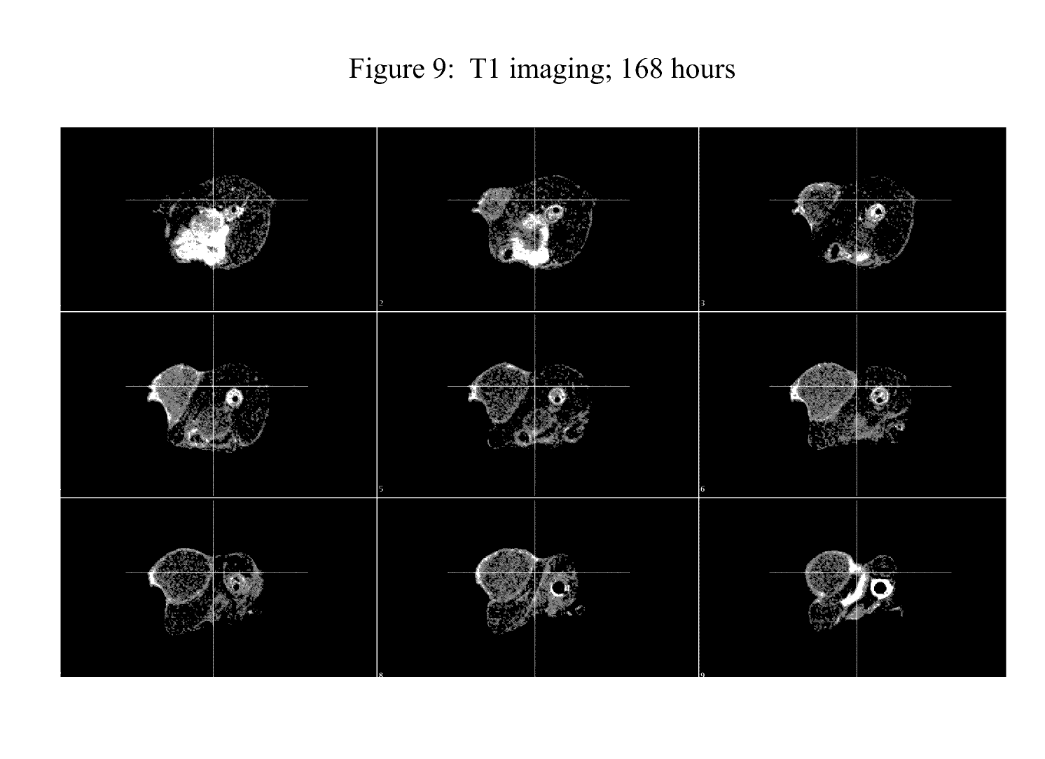

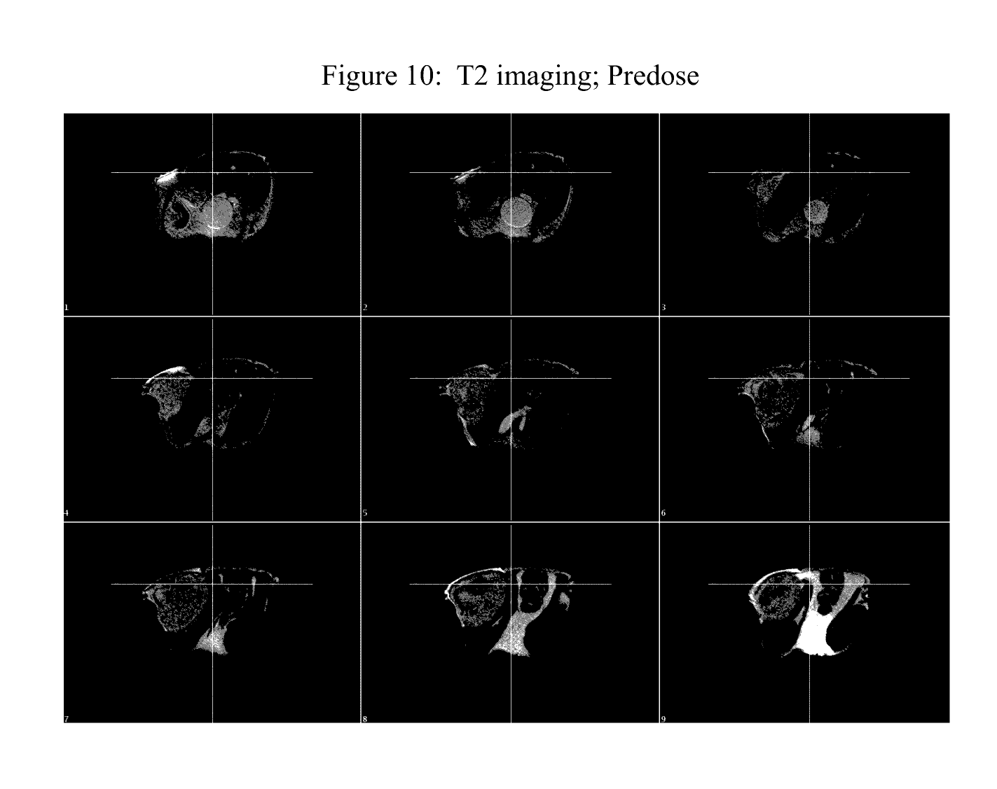

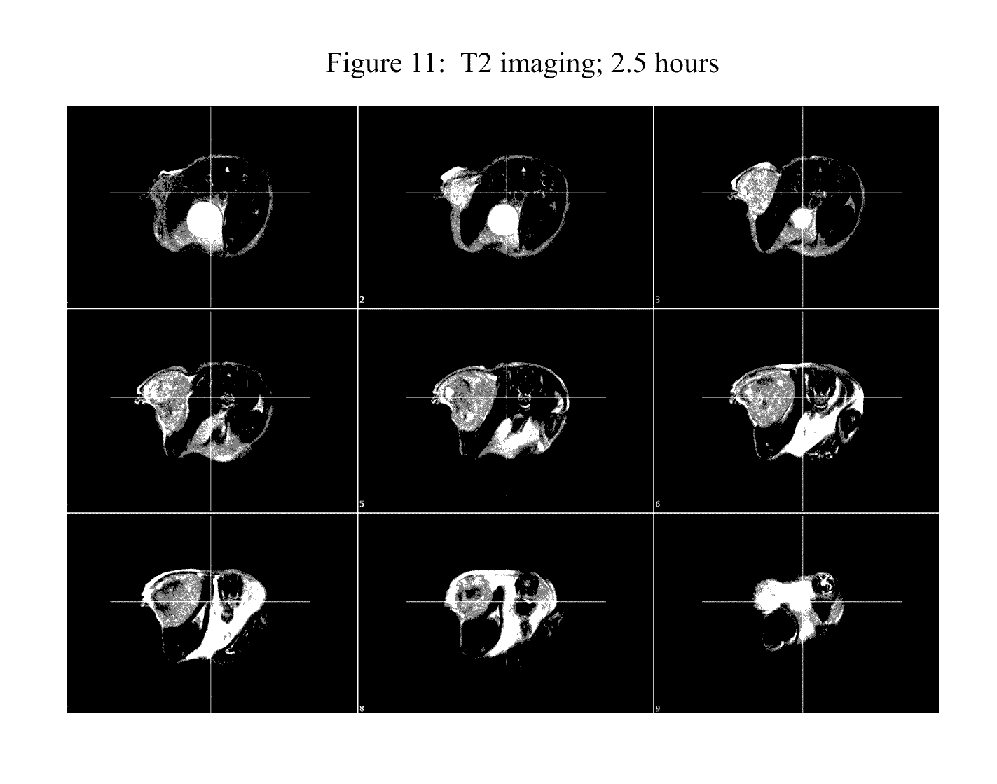

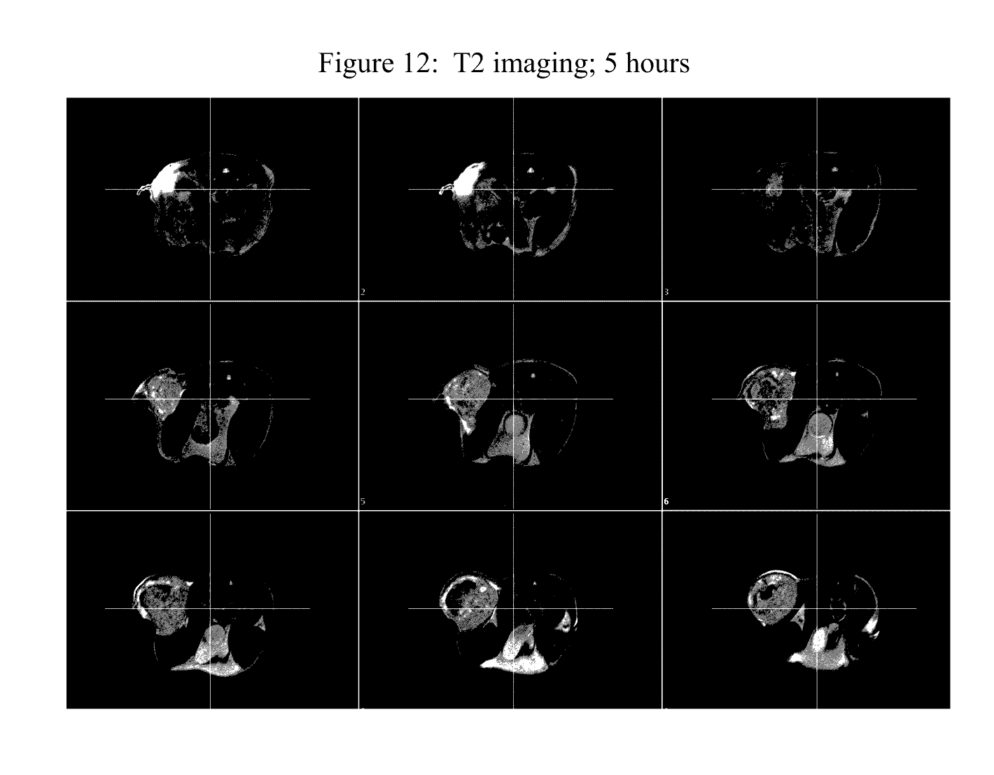

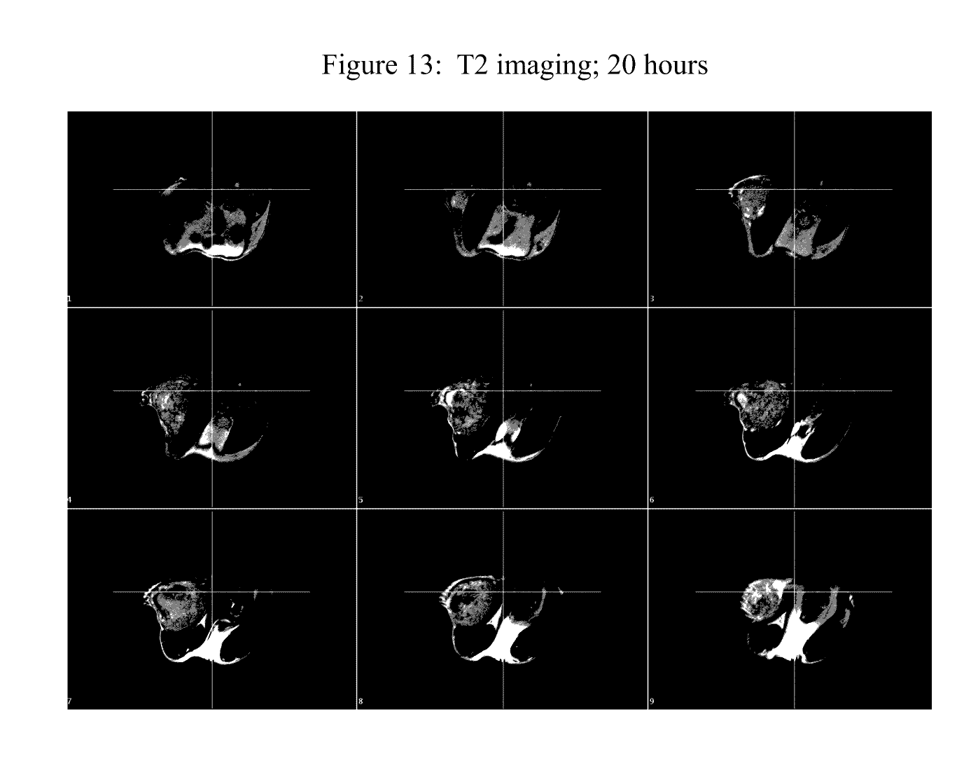

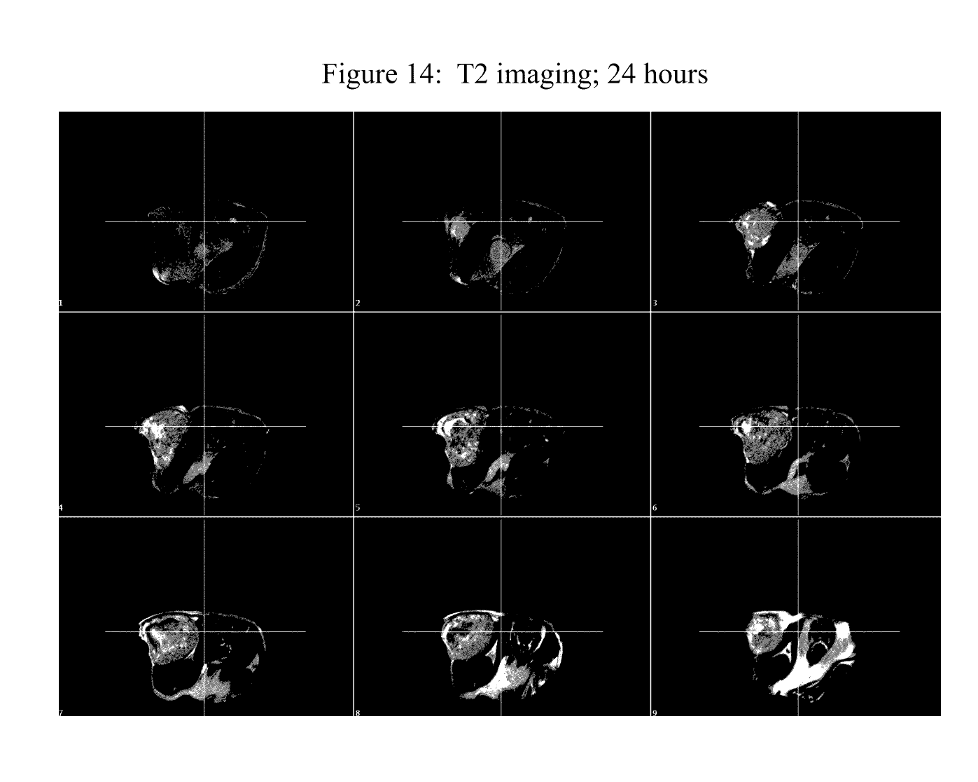

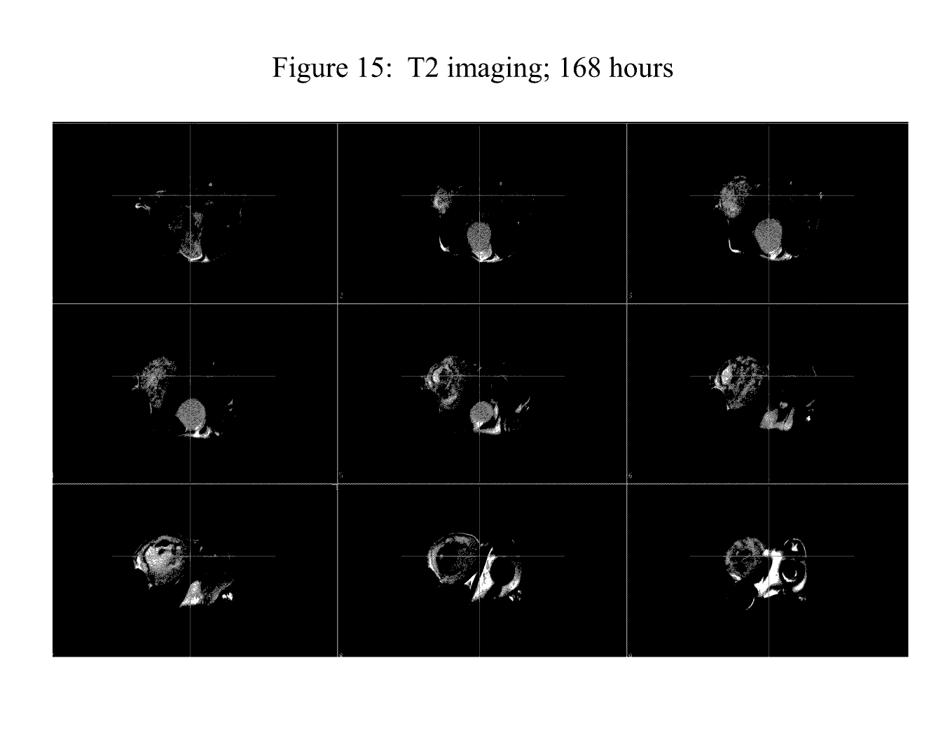

MRI imaging of aHCT 116 cell line human colon cancer xenograft mouse was performed using a 7T Varian small animal MRI. SN-38 loaded, iron stabilized micelles were administered by tail vein injection. The animal was serially imaged with both T1 weighted and T2 weighted imaging sequences prior to dosing and 2.5, 5, 20, 24 and 168 hours after administration of the SN-38 loaded, iron stabilized micelles. FIG. 4 shows the T1 weighted imaging, at different depths, prior to dosing. FIG. 5 shows the T1 weighted imaging after 2.5 hours. FIG. 6 shows the T1 weighted imaging after 5 hours. FIG. 7 shows the T1 weighted imaging after 20 hours. FIG. 8 shows the T1 weighted imaging after 24 hours. FIG. 9 shows the T1 weighted imaging after 168 hours. FIG. 10 shows the T2 weighted imaging prior to dosing. FIG. 11 shows the T2 weighted imaging after 2.5 hours. FIG. 12 shows the T2 weighted imaging after 5 hours. FIG. 13 shows the T2 weighted imaging after 20 hours. FIG. 14 shows the T2 weighted imaging after 24 hours. FIG. 15 shows the T2 weighted imaging after 168 hours. Each of the figures depicting different axial (transverse) cross-sections of the animal, taken at different depths. The tumor is in shown in the upper left of the animal cross-section. As can be shown in the images, enhanced contrast can be seen in the tumor environment at 2.5, 5, 20, and 24 hours after administration when compared to the predose and 168 hour images. One skilled in the art can appreciate that because individual iron ions or chelates do not provide contrast in MRI imaging, the contrast appearing in the tumor is due to the accumulation of intact polymer micelles. One skilled in the art will also appreciate that the contrast imparted by the nanoparticles has dissipated by 168 hours.

### Example 3

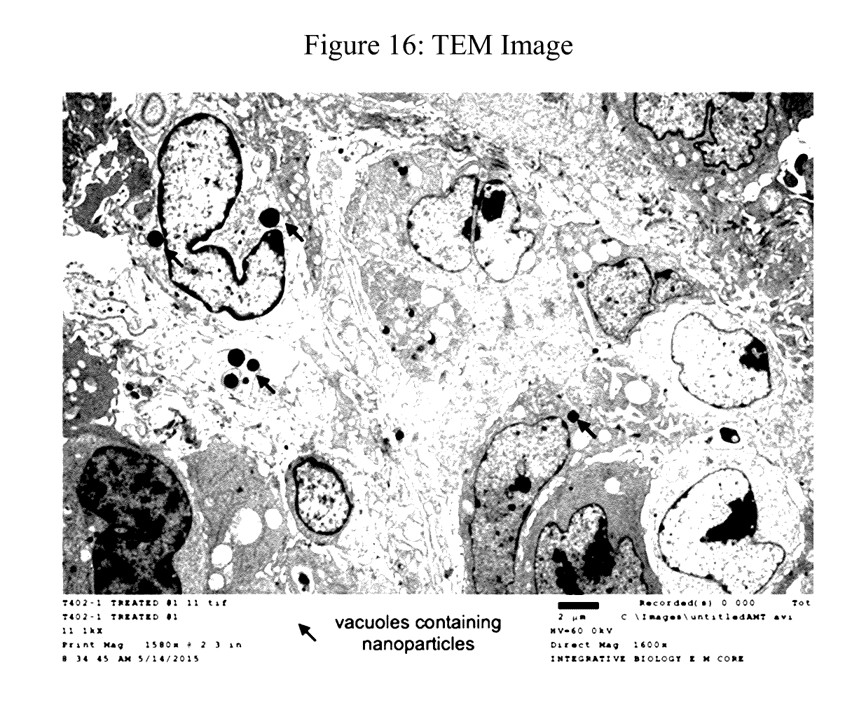

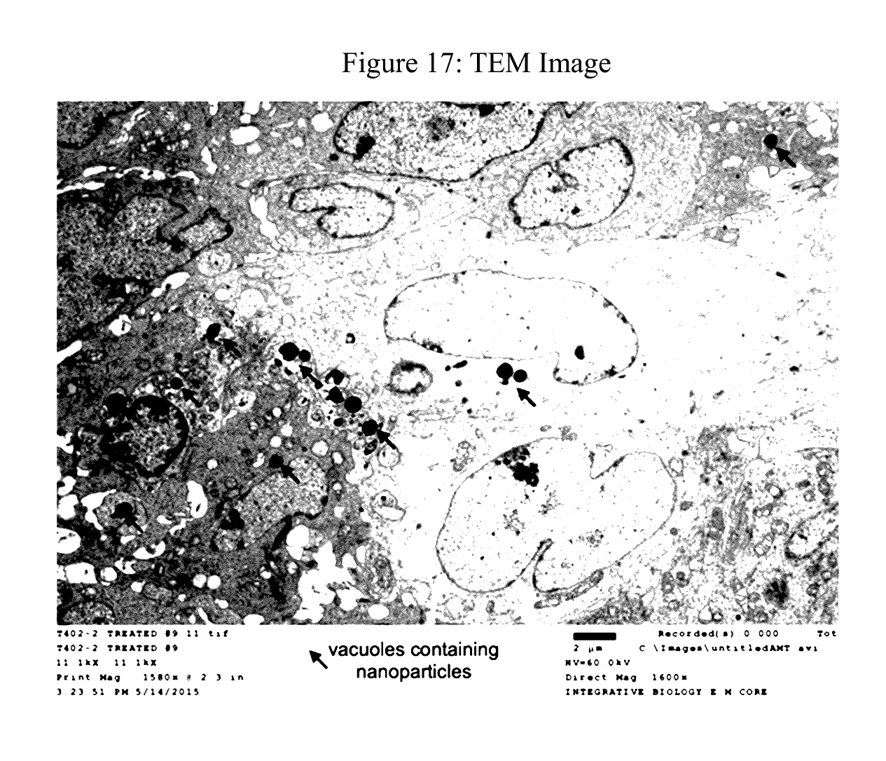

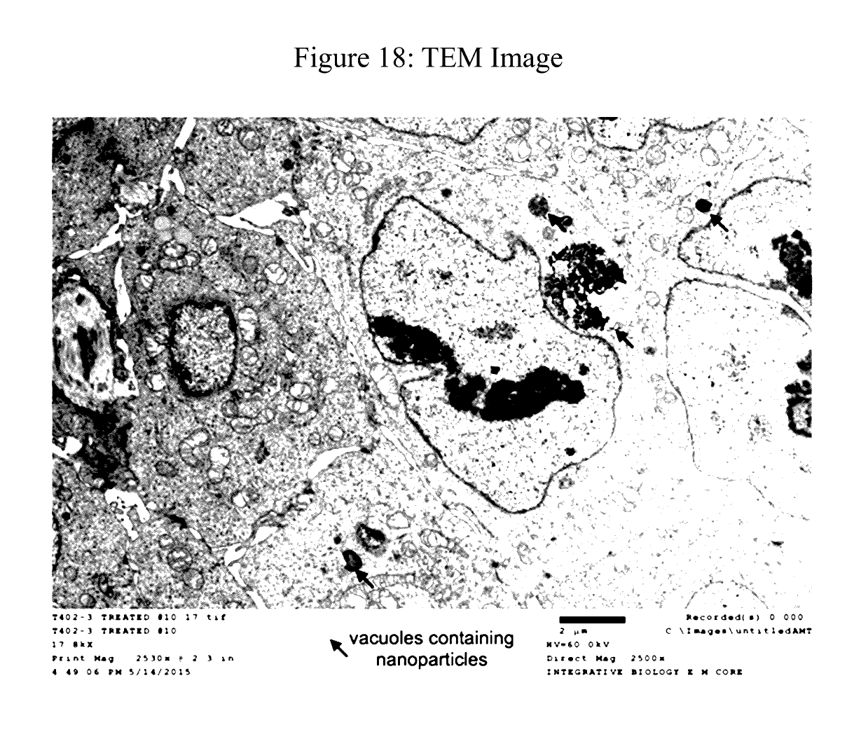

Transmission electron microscopy was performed on HCT-116 cell line human colon cancer xenograft mouse tissue. SN-38 loaded, iron stabilized micelles were administered by tail vein injection to a mouse possessing an HCT-116 human colon cancer xenograft tumor. After 1 hour, the animal was sacrificed, and the tumor tissue collected. The tumor tissued was fixed, cut into 70-80 nm thick sections with a microtome, then stained with osmium tetroxide, lead citrate, and uranyl acetate for microscopy. Cross sections were placed on a copper grid then imaged with a transmission electron microscope. Representative images are shown in FIG. 16, FIG. 17, and FIG. 18. Arrows indicate the presence of vacuoles that contain SN-38 loaded, iron stabilized micelles. One skilled in the art will appreciate that these images indicate that the micelles are taken into tumor cells and tumor macrophages while they are intact (e.g. micelles accumulate in the tumor, then are taken up as a micellar nanoparticle into the tumor cells). One skilled in the art will also appreciate that the vacuoles expand as they reach late endosome stage, as seen in FIG. 18.

### Example 4

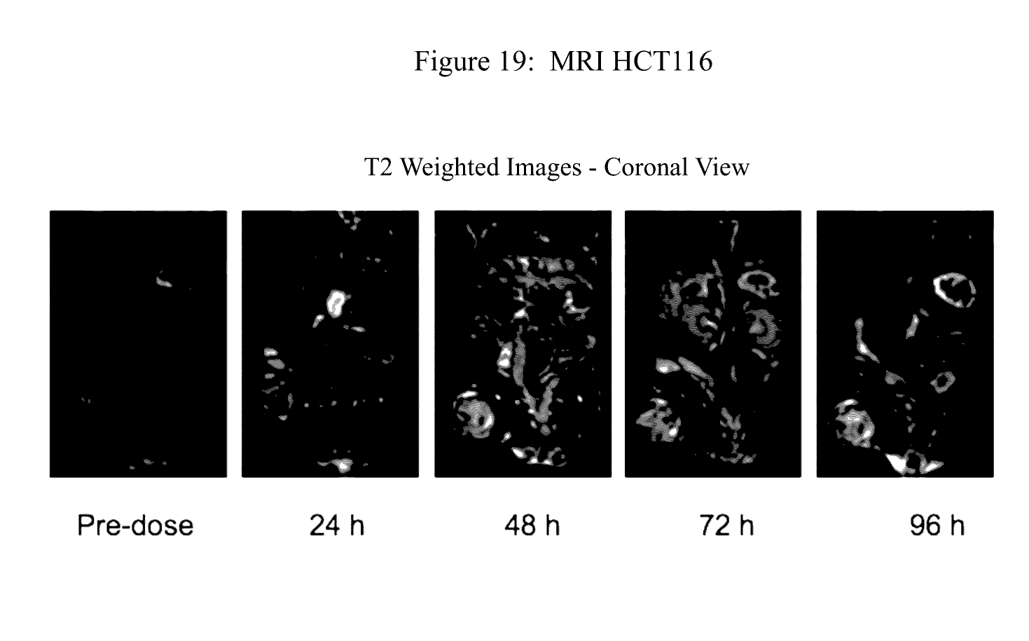

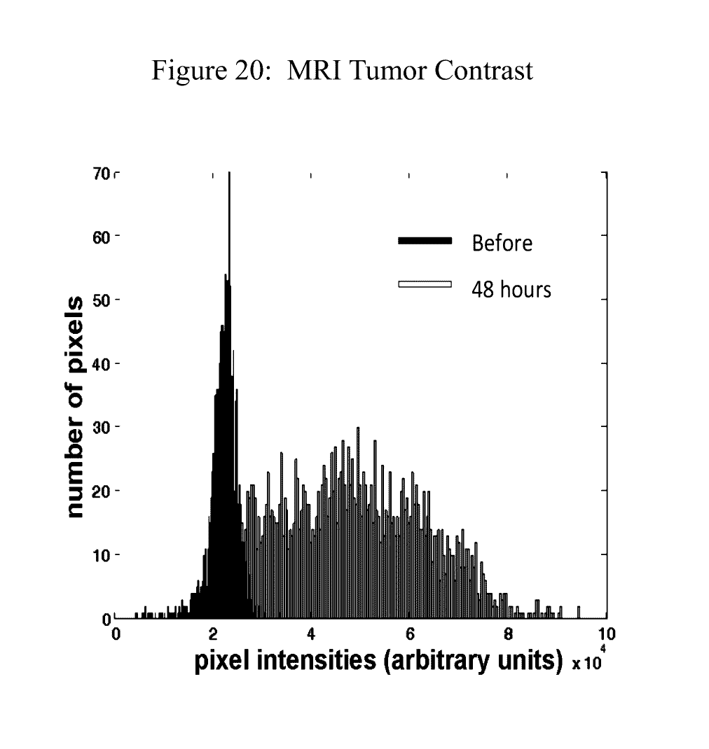

MRI imaging of a HCT-116 cell line human colon cancer xenograft mouse was performed using a 7T Varian small animal MRI. SN-38 loaded, iron stabilized micelles were administered by tail vein injection. The animal was serially imaged with both T1 weighted and T2 weighted imaging sequences prior to dosing and 24, 48, 72, and 96 hours after administration of the SN-38 loaded, iron stabilized micelles. FIG. 19 shows a time course of the coronal images. The tumor is in shown in the lower left of each image. Enhanced contrast can be seen in the tumor environment at 24, 48, 72, and 96 hours after administration when compared to the predose image. One skilled in the art can appreciate that because individual iron ions or chelates do not provide contrast in MRI imaging, the contrast appearing in the tumor is due to the accumulation of intact polymer micelles. FIG. 20 depicts a histogram of contrast in the tumor ROI predose and at 24 hours.

### Example 5

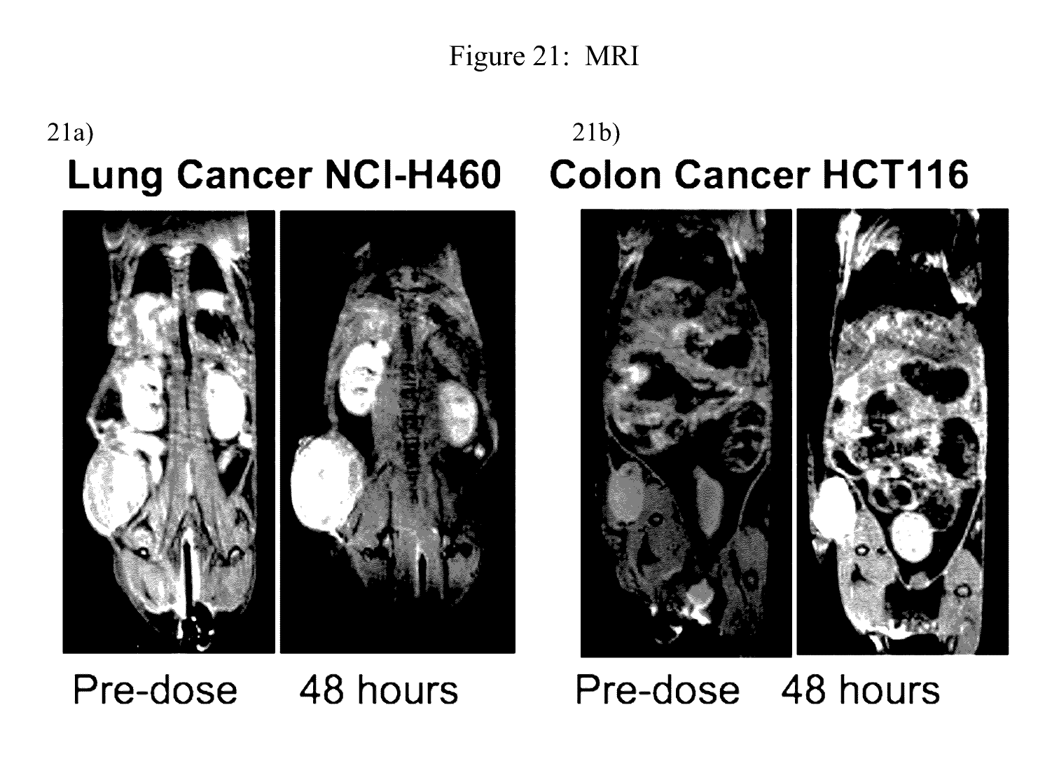

MRI imaging of a HCT-116 cell line human colon cancer xenograft mouse and an NCI-H460 lung cancer xenograft mouse was performed using a 7T Varian small animal MRI. Epothilone D loaded, iron stabilized micelles were administered by tail vein injection. The animal was serially imaged with both T1 weighted and T2 weighted imaging sequences prior to dosing and 48 hours after administration of the epothilone D loaded, iron stabilized micelles. FIG. 21a shows the MR image pre-dose and 48 hours post dosing of epothilone D loaded, iron stabilized micelles in lung cancer NCI-H460 xenograft mouse. FIG. 21b shows the MR image pre-dose and 48 hours post dosing of epothilone D loaded, iron stabilized micelles in human colon cancer HCT-116 cell line xenograft mouse. The tumor is in shown in the lower left of each image. Enhanced contrast can be seen in the tumor environment at 48 hours after administration when compared to the predose image. One skilled in the art can appreciate that because individual iron ions or chelates do not provide contrast in MRI imaging, the contrast appearing in the tumor is due to the accumulation of intact polymer micelles.

We claim:

1. A diagnostic imaging method comprising the steps of: (a) administering to a subject a provided drug loaded, iron stabilized micelles, or composition thereof; and (b) imaging the iron stabilized micelles after administration to the subject by magnetic resonance imaging.

2. A method of treating a subject with cancer comprising the following steps: 1) administration of drug loaded, iron stabilized micelles, or composition thereof, to a subject possessing a solid tumor malignancy; 2) imaging said tumor with magnetic resonance imaging; 3) confirming that contrast is observed in the tumor; and 4) continuing treatment schedule.
